(19) United States
(12) Patent Application Publication
(10) Pub. No.: US 2020/0375549 A1
(43) Pub. Date: Dec. 3, 2020

(54) SYSTEMS FOR BIOMONITORING AND BLOOD GLUCOSE FORECASTING, AND ASSOCIATED METHODS

(71) Applicant: INFORMED DATA SYSTEMS INC. D/B/A ONE DROP, New York, NY (US)

(72) Inventors: Ydo Wexler, Haifa (IL); Daniel R. Goldner, Minnetonka, MN (US); Jeffrey Dachis, Brooklyn, NY (US)

(21) Appl. No.: 16/888,105

(22) Filed: May 29, 2020

Related U.S. Application Data
(60) Provisional application No. 62/855,194, filed on May 31, 2019.
(60) Provisional application No. 62/981,914, filed on Feb. 26, 2020.

(51) Int. Cl. A61B 5/00; A61B 5/145; A61B 5/11; G16H 10/60; G16H 50/20; G16H 50/30; G16H 50/70; G16H 40/67; G16H 15/00

(52) U.S. Cl. CPC A61B 5/7275; A61B 5/7475; A61B 5/7267; A61B 5/4848; A61B 5/1118; A61B 5/4866; G16H 10/60; G16H 50/20; G16H 50/30; G16H 50/70; G16H 40/67; G16H 15/00; A61B 5/0004; A61B 5/7435; A61B 5/14532

(57) ABSTRACT

Systems and methods for biomonitoring and personalized healthcare are disclosed herein. In some embodiments, a computer-implemented method for forecasting a blood glucose state of a patient is provided. The method comprises: receiving blood glucose data of the patient; generating at least one initial prediction of the blood glucose state by inputting the blood glucose data into a first set of machine learning models; determining a plurality of features at least partly from the at least one initial prediction; and generating a final prediction of the blood glucose state by inputting the plurality of features into a second set of machine learning models.

### CROSS-REFERENCE TO RELATED APPLICATIONS

The present application claims the benefit of U.S. Provisional Patent Application No. 62/855,194, filed May 31, 2019, entitled CONTINUOUS BLOOD GLUCOSE MONITORING, and U.S. Provisional Patent Application No. 62/981,914, filed Feb. 26, 2020, entitled HYPOGLYCEMIA PREDICTION, all of which are incorporated by reference herein in their entireties.

The present application is related to U.S. patent application Ser. No. 16/558,558, filed Sep. 3, 2019, entitled FORECASTING BLOOD GLUCOSE CONCENTRATION, which is incorporated herein by reference in its entirety.

### TECHNICAL FIELD

This disclosure relates generally to personalized healthcare and, in particular, to systems and methods for biomonitoring and forecasting a patient's blood glucose state.

### BACKGROUND

Diabetes mellitus (DM) is a group of metabolic disorders characterized by high blood glucose levels over a prolonged period. Typical symptoms of such conditions include frequent urination, increased thirst, increased hunger, etc. If left untreated, diabetes can cause many complications. There are three main types of diabetes: Type 1 diabetes, Type 2 diabetes, and gestational diabetes. Type 1 diabetes results from the pancreas' failure to produce enough insulin. In Type 2 diabetes, cells fail to respond to insulin properly. Gestational diabetes occurs when pregnant women without a previous history of diabetes develop high blood glucose levels.

Diabetes affects a significant percentage of the world's population. Timely and proper diagnoses and treatment are essential to maintaining a relatively healthy lifestyle for individuals with diabetes. Application of treatment typically relies on accurate determination of glucose concentration in the blood of an individual at a present time and/or in the future. However, conventional blood glucose monitoring systems may be unable to provide real-time analytics, personalized analytics, or blood glucose concentration forecasting, or may not provide such information in a rapid, reliable, and accurate manner. Thus, there is a need for improved systems and methods for biomonitoring and/or providing personalized healthcare recommendations or information for the treatment of diabetes and associated conditions.

### BRIEF DESCRIPTION OF THE DRAWINGS

FIG. 1 is a schematic diagram of an exemplary computing environment in which a biomonitoring and forecasting system operates, in accordance with embodiments of the present technology.

FIG. 2 is a flowchart illustrating a method for preparing blood glucose data for use in biomonitoring and forecasting, in accordance with embodiments of the present technology.

FIG. 3 is a block diagram illustrating a method for forecasting a blood glucose state of a patient, in accordance with embodiments of the present technology.

FIG. 4 is a schematic block diagram illustrating a machine learning architecture for blood glucose forecasting configured in accordance with embodiments of the present technology.

FIG. 5 is a block diagram illustrating a method for forecasting an overnight hypoglycemia event, in accordance with embodiments of the present technology.

FIGS. 6A-6I illustrate various graphical user interfaces configured in accordance with embodiments of the present technology.

FIG. 7 is a schematic block diagram of a computing system or device configured in accordance with embodiments of the present technology.

FIG. 8 is a graph illustrating exemplary sequences of nightly probabilities of overnight hypoglycemia for selected individuals.

### DETAILED DESCRIPTION

The present technology generally relates to systems and methods for biomonitoring and providing personalized healthcare. In some embodiments, the systems and methods herein are configured to forecast or predict various aspects of a patient's health at a future time point or period, such as a blood glucose state (e.g., a blood glucose level, likelihood of a hypoglycemic or hyperglycemic event, etc.). For example, a computer-implemented method for forecasting or predicting a patient's blood glucose state can include receiving blood glucose data of the patient (e.g., a plurality of blood glucose measurements from a continuous glucose monitoring (CGM) device). The blood glucose data can be correlated with at least one event (e.g., insulin intake, meal intake, physical activity, etc.). The method can include generating at least one initial prediction of the blood glucose state by inputting the blood glucose data into a first set of machine learning models. The method can also include determining a plurality of features from the at least one initial prediction, and optionally from other patient data (e.g., the blood glucose data, previous blood glucose data, personal data, etc.). The method can further include generating a final prediction of the blood glucose state by inputting the plurality of features into a second set of machine learning models. The systems and methods described herein can rapidly and accurately predict a patient's future blood glucose state, even in situations where the data for that patient is limited, irregular, and/or incomplete. Accordingly, the present technology can be used to provide personalized notifications, feedback, and/or recommendations in real time to improve health outcomes of patients with diabetes and related conditions.

### Overview of Technology

Oscillations in levels of blood glucose in the human body are a natural result of a complex mechanism, the main effect of which may be due to the changing balance between food consumed, especially carbohydrates, and insulin, which regulates the metabolism of carbohydrates, fats, and protein in the body. Although the effect of this balance and other factors may be unique to each individual, common biological, physical, and sociological patterns between individuals make observations of the changes in blood glucose levels valuable to assessing the expected changes in other people.

Two special conditions that may occur with fluctuations in blood glucose levels are hyperglycemia and hypoglycemia. Hyperglycemia, or high blood glucose, is a condition in which an excessive amount of glucose circulates in the blood plasma. This is generally a blood glucose level higher than 180 mg/dL. Hypoglycemia, or low blood glucose, is a condition in which blood glucose levels decrease below normal levels. Most individuals feel symptoms of hypoglycemia when their blood glucose level is 70 mg/dL or lower. The symptoms usually include hunger, shakiness, anxiety, sweating, pale skin, fast or irregular heartbeat, sleepiness, dizziness, crankiness, clumsiness, etc. If left untreated, the symptoms can become worse and may include confusion, trouble talking, blurred vision, passing out, loss of consciousness, seizures, or even death. Hypoglycemia is most common in diabetic patients who may have issues with medicine, food, exercise, etc. Individuals with diabetes may also experience hypoglycemia events as a result of medications (e.g., insulin, sulfonylureas, etc.) that they may be taking for their condition. However, even individuals who do not have diabetes can experience hypoglycemia.

Accordingly, the present technology can include methods, systems, articles of manufacture, and the like that can, among other possible advantages, provide a way to recast and interpret blood glucose data and other data related to the patient, which may include data resulting from continuous blood glucose monitoring, for the purposes of predicting blood glucose levels and/or an occurrence of a hyperglycemic event or hypoglycemic event (or any other event) during a predetermined period of time (e.g., within the next 15 minutes, 30 minutes, 60 minutes, 90 minutes, 2 hours, 4 hours, or overnight).

In some embodiments, the present technology relates to a computer-implemented system, method, and/or a computer program product that may be configured to forecast, at any moment, values of future blood glucose levels of an individual up to a certain point in time, and in addition, to predict the probability of blood glucose concentration rising and/or dropping (e.g., beyond a certain threshold) within a certain time period (e.g., to determine whether hypoglycemia, hyperglycemia, and/or any other medical condition may occur).

In some embodiments, the present technology may rely on the fact that various complex mechanisms may determine blood glucose levels in a body of the user, and may therefore implement a suitable model or models that receives, generalizes, and/or otherwise processes information involved in such mechanisms. In some embodiments, once the model(s) are defined, the present technology may generate predictions without obtaining blood glucose levels constantly and/or without knowledge of the current glucose levels of other individuals.

In some embodiments, the present technology provides a computing system and/or framework for performing such determining, forecasting, and/or interpretation of input data, such as blood glucose data and/or other data related to the patient. The input data can include at least one of the following: current and/or previous blood glucose measurement data of the patient, current and/or previous blood glucose measurement data of other patients (e.g., the data can be appropriately anonymized), data resulting from continuous monitoring of blood glucose concentration, and/or any other data related to blood glucose concentrations, meal characteristics data (e.g., number of meals, time of meals, grams carbohydrates consumed during meal times (whether currently and/or in the past)), blood pressure data, sleeping patterns data, heart rate data, physical activity data (e.g., workout times, activity type (e.g., walking, running, etc.), current and/or previous weight data of the patient, current and/or previous al c data values, personal data and/or medical history data related to the patient (e.g., diabetes type, family history, patient health history, diagnoses, blood pressure, age, gender, demographics, etc.), as well as similar types of data related to other patients. One or more of the above data may be collected in real-time, continuously, during predetermined periods of time, periodically (e.g., at certain preset periods of time, e.g., every 5 minutes, every hour, etc.). The data may be queried upon execution of certain processes of the methods described herein.

In some embodiments, the systems herein may be configured to predict an expected blood glucose level or concentration based on one or more past observations of an individual or patient whose blood glucose concentration is being predicted, one or more observations of blood glucose concentrations along with other information reported from a multitude of individuals, and/or continuous monitoring data, and/or any combination thereof. The data considered in predicting blood glucose concentration may include personal data such as gender and year of diagnosis, historical blood glucose data, and/or any other self-reported, health-related data including food, medications, exercise and/or any other data, and/or any combination thereof.

As stated above, the current technology may also incorporate data collected from a CGM device or component that may continuously provide (e.g., determine and/or transmit) blood glucose concentration data using various time intervals (e.g. every 5 minutes). The intervals may be predetermined, arbitrary, preset based on a specific monitoring schedule for the user and/or condition, and/or determined in any other fashion.

In some embodiments, for example, the current technology may be configured to generate one or more of the following types of predictions that may incorporate CGM data as inputs to the predictive model(s):
predicting blood glucose level(s) of an individual up to a certain time at fixed intervals (e.g., 60 minutes in advance);
predicting a probability that an individual will experience hyperglycemia (e.g., blood-glucose levels above 180 mg/dL) within a defined time frame in the future (e.g., in the next 60 minutes, or during the interval from 30-90 minutes from now); and/or
predicting a probability that an individual will experience hypoglycemia (e.g., blood-glucose levels below 70 mg/dL) within a defined time frame in the future.

The techniques described herein for continuous glucose monitoring and forecasting may provide for real-time feedback to the monitored individual either directly and/or indirectly, and hence, allow for educated decisions in the everyday management of the individual's health conditions.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The headings provided herein are for convenience only and do not interpret the scope or meaning of the claimed present technology.

### Systems and Methods for Biomonitoring and Blood Glucose Forecasting

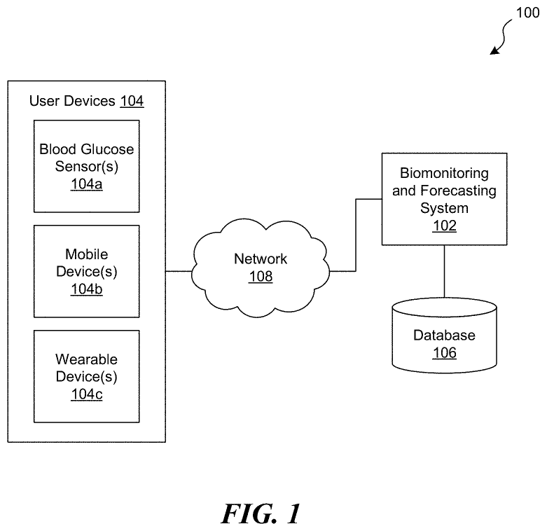

FIG. 1 is a schematic diagram of an exemplary computing environment 100 in which a biomonitoring and forecasting system 102 (“system 102”) operates, in accordance with embodiments of the present technology. As shown in FIG. 1, the system 102 is operably couple to one or more user devices 104 via a network 108. The system 102 is also operably coupled to at least one database or storage component 106 (“database 106”). The system 102 can be configured to monitor and predict a patient's blood glucose state, as described in greater detail below. The blood glucose state can be any status, condition, parameter, etc. that is associated with or otherwise related to the patient's blood glucose. For example, the blood glucose state can include the patient's blood glucose level, whether the patient is hypoglycemic, whether the patient is hyperglycemic, etc. In some embodiments, the system 102 receives input data (e.g., blood glucose data and/or other data) and performs monitoring, processing, analysis, forecasting, interpretation, etc. of the input data in order to generate predictions of the patient's blood glucose state. The system 102 can also be configured to output notifications, recommendations, and/or other information to the patient based on the predicted blood glucose state.

The system 102 can include processors, memory, and/or other software and/or hardware components configured to implement the various methods described herein. For example, the system 102 can be or include a forecasting and/or analysis engine having a CGM component that predicts a patient's blood glucose level based on CGM data. Optionally, the forecasting and/or analysis engine can also include a hypoglycemic event prediction component that predicts whether the patient is likely to experience an overnight hypoglycemic event, as discussed in greater detail below.

In some embodiments, the system 102 receives input data from one or more user devices 104. The user devices 104 can be any device associated with a patient or other user, and can be used to obtain blood glucose data and/or any other relevant input data (e.g., health data, food data, medication data, physical activity data, etc.) relating to the patient and/or any other users or patients (e.g., appropriately anonymized patient data). In the illustrated embodiment, for example, the user devices 104 include at least one blood glucose sensor 104a, at least one mobile device 104b (e.g., a smartphone or tablet computer), and, optionally, at least one wearable device 104c (e.g., a smartwatch). In other embodiments, however, one or more of the devices 104a-c can be omitted and/or other types of user devices can be included (e.g., computing devices such as personal computers, laptop computers, etc.; biomonitoring devices such as blood pressure sensors, heart rate sensors, sleep trackers, temperature sensors, etc.). Additionally, although FIG. 1 illustrates the blood glucose sensor(s) 104a as being separate from the other user devices 104, in other embodiments the blood glucose sensor(s) 104a can be incorporated into another user device 104.

The blood glucose sensor(s) 104a can include any device capable of obtaining blood glucose data from the patient, such as implanted sensors, non-implanted sensors, invasive sensors, minimally invasive sensors, non-invasive sensors, wearable sensors, etc. The blood glucose sensor(s) 104a can be configured to obtain samples from the patient (e.g., blood samples) and determine glucose levels in the sample. Any suitable technique for obtaining patient samples and/or determining glucose levels in the samples can be used. In some embodiments, for example, the blood glucose sensor(s) 104a can be configured to detect substances (e.g., a substance indicative of glucose levels), measure a concentration of glucose, and/or measure another substance indicative of the concentration of glucose. The blood glucose sensor(s) 104a can be configured to analyze, for example, body fluids (e.g., blood, interstitial fluid, sweat, etc.), tissue (e.g., optical characteristics of body structures, anatomical features, skin, or body fluids), and/or vitals (e.g., heat rate, blood pressure, etc.) to periodically or continuously obtain blood glucose data. Optionally, the blood glucose sensor(s) 104a can include other capabilities, such as processing, transmitting, receiving, and/or other computing capabilities.

The blood glucose sensor(s) 104a can include various types of sensors, such as chemical sensors, electrochemical sensors, optical sensors (e.g., optical enzymatic sensors, opto-chemical sensors, fluorescence-based sensors, etc.), spectrophotometric sensors, spectroscopic sensors, polarimetric sensors, calorimetric sensors, iontophoretic sensors, radiometric sensors, and the like, and combinations thereof. In some embodiments, the blood glucose sensor(s) 104a include at least one CGM device or sensor that measures the patient's blood glucose level at predetermined time intervals. For example, the CGM device can obtain at least one blood glucose measurement every minute, 2 minutes, 5 minutes, 10 minutes, 15 minutes, 20 minutes, 30 minutes, 60 minutes, 2 hours, etc. In some embodiments, the time interval is within a range from 5 minutes to 10 minutes.

Optionally, the blood glucose sensor(s) 104a or another user device 104 can be configured to obtain various measurements, statistics, and/or transformations associated with a number of past blood glucose measurements. For example, a quadratic fit (e.g., intercept, first order coefficient, second order coefficient) to the past number of (e.g., 24) blood glucose measurements (e.g., past 2 hours) may be obtained. The quadratic fit to the past number of blood glucose measurements may be selected over a linear or cubic fit to achieve the highest forecast accuracy. As another example, averages and/or standard deviations of past blood glucose measurements may be obtained (e.g., over the past 24 hours, over all past measurements, etc.).

The user devices 104 can also include one or more devices that allow for entry of additional types of data, such as meal or nutrition data (e.g., number of meals; timing of meals; number of calories; amount of carbohydrates, fats, sugars, etc.), medical history or health data (e.g., weight, age, sleeping patterns, medical conditions, cholesterol levels, diabetes type, family history, patient health history, diagnoses, blood pressure, etc.), physical activity or exercise data (e.g., time and/or duration of activity; activity type such as walking, running, swimming; strenuousness of the activity such as low, moderate, high; etc.), personal data (e.g., name, gender, demographics, social network information, etc.), medication data (e.g., timing and/or dosages of medications such as insulin), and/or any other data, and/or any combination thereof.

In some embodiments, one or more of the user devices 104 can be configured to obtain other physiological data of the patient, such as cardiovascular data, respiratory data, body temperature data (e.g., skin temperature data), sleep data, stress level data (e.g., cortisol and/or other chemical indicators of stress levels), al c data, biomarker data (e.g., for other diseases or conditions), and/or data of any other suitable physiological parameters. For example, cardiovascular data can include any physiological parameter related to the patient's cardiovascular health, such as blood pressure data, heart rate data, arrhythmia event data (if any), pacemaker data, etc. In some embodiments, the cardiovascular data can be the “most recent” data, e.g., data taken within the last minute, 2 minutes, 5 minutes, 10 minutes, 15 minutes, 20 minutes, 30 minutes, 60 minutes, 2 hours, etc. For example, the blood pressure data can include the most recent systolic and/or diastolic blood pressure measurement(s) of the patient. By way of a non-limiting example, the most recent systolic blood pressure measurements may improve forecast accuracy more than other types of blood pressure measurements (e.g., most recent diastolic blood pressure, average systolic blood pressure, etc.).

As another example, sleep data can include any parameter relevant to the patient's sleep habits, such as the number of hours of sleep, average hours of sleep, variability of hours of sleep, sleep-wake cycle data, data related to sleep apnea events (if any), sleep fragmentation (e.g., fraction of nighttime hours awake between sleep episodes, etc.), frequency of low blood glucose concentration (e.g., <70 mg/dL) while the patient is sleeping, etc. during one or more previous nights. For example, the previous night(s)' sleep data may be configured to improve forecast accuracy and may be used to determine sleep-hour statistics, which may include previous frequency of overnight hypoglycemia. The sleep data may also be used to identify “bedtimes” (e.g., beginning of each night's sleep), e.g., in order to identify forecast times and/or actual overnight hypoglycemia events that may be used for testing and/or training, as discussed below. In some embodiments, the sleep data is used exclusively for overnight hypoglycemia prediction, as described further below.

In some embodiments, some or all of the user devices 104 are configured to continuously obtain any of the above data (including blood glucose concentrations, health data, etc.) from the patient over a particular time period (e.g., hours, days, weeks, months, years). For example, data can be obtained at a predetermined time interval (e.g., once every minute, 2 minutes, 5 minutes, 10 minutes, 15 minutes, 20 minutes, 30 minutes, 60 minutes, 2 hours, etc.), at random time intervals, or combinations thereof. The time interval for data collection may be relatively short compared to the time period for which a forecast or prediction is to be made (e.g., 1 to 2 hours in the future). The time interval for data collection can be set by the patient, by another user (e.g., a physician), by the system 102, or by the user device 104 itself (e.g., as part of an automated data collection program). The user device 104 can obtain the data automatically or semi-automatically (e.g., by automatically prompting the patient to provide such data at a particular time), or from manual input by the patient (e.g., without prompts from the user device 104). The continuous data may be provided to the system 102 at predetermined time intervals (e.g., once every minute, 2 minutes, 5 minutes, 10 minutes, 15 minutes, 20 minutes, 30 minutes, 60 minutes, 2 hours, etc.), continuously, in real-time, upon receiving a query, manually, automatically (e.g., upon detection of new data), semi-automatically, etc. The time interval at which the user device 104 obtains data may or may not be the same as the time interval at which the user device 104 transmit the data to the system 102.

The user devices 104 can obtain any of the above data in various ways, such as using one or more of the following components: a microphone (either a separate microphone or a microphone imbedded in the device), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, a sensor (e.g., a sensor included in or operably coupled to the user device 104), and/or any other device. The data obtained by the user devices 104 can include metadata, structured content data, unstructured content data, embedded data, nested data, hard disk data, memory card data, cellular telephone memory data, smartphone memory data, main memory images and/or data, forensic containers, zip files, files, memory images, and/or any other data/information. The data can be in various formats, such as text, numerical, alpha-numerical, hierarchically arranged data, table data, email messages, text files, video, audio, graphics, etc. Optionally, any of the above data can be filtered, smoothed, augmented, annotated, or otherwise processed (e.g., by the user devices 104 and/or the system 102) before being used for analysis and/or forecasting, as described in greater detail below.

In some embodiments, any of the above data can be queried by one or more of the user devices 104 from one or more databases (e.g., the database 106, a third-party database, etc.). The user device 104 can generate a query and transmit the query to the system 102, which can determine which database may contain requisite information and then connect with that database to execute a query and retrieve appropriate information. In other embodiments, the user device 104 can receive the data directly from the third-party database and transmit the received data to the system 102, or can instruct the third-party database to transmit the data to the system 102. In some embodiments, the system 102 can include various application programming interfaces (APIs) and/or communication interfaces that can allow interfacing between user devices 104, databases, and/or any other components.

Optionally, the system 102 can also obtain any of the above data from various third party sources, e.g., with or without a query initiated by a user device 104. In some embodiments, the system 102 can be communicatively coupled to various public and/or private databases that can store various information, such as census information, health statistics (e.g., appropriately anonymized), demographic information, population information, and/or any other information. For example, the system 102 can obtain information about blood glucose levels and/or forecasts of blood glucose levels of a plurality of users (e.g., without identifying the users) of the system 102, nutrition data relating to such users, exercise data, social network information, and/or any other information and/or any combination thereof, as described in greater detail below. Additionally, the system 102 can also execute a query or other command to obtain data from the user devices 104 and/or access data stored in the database 106. The data can include data related to the particular patient and/or a plurality of patients or other users (e.g., historical blood glucose concentration levels, prior analyses of blood glucose measurements, health history data, medical condition history data, exercise history data, nutrition data, etc.), as described herein.

The database 106 can be used to store various types of data obtained and/or used by the system 102. For example, any of the above data can be stored in the database 106. The database 106 can also be used to store data generated by the system 102, such as previous predictions or forecasts produced by the system 102. In some embodiments, the database 106 includes data for multiple users, such as a plurality of patients (e.g., at least 50, 100, 200, 500, 1000, 2000, 3000, 4000, 5000, or 10,000 different patients). The data can be appropriately anonymized to ensure compliance with various privacy standards. The database 106 can store information in various formats, such as table format, column-row format, key-value format, etc. (e.g., each key can be indicative of various attributes associated with the user and each corresponding value can be indicative of the attribute's value (e.g., measurement, time, etc.)). In some embodiments, the database 106 can store a plurality of tables that can be accessed through queries generated by the system 102 and/or the user devices 104. The tables can store different types of information (e.g., one table can store blood glucose measurement data, another table can store user health data, etc.), where one table can be updated as a result of an update to another table.

For example, Table 1 below illustrates exemplary health and/or behavioral data that may be provided to the system 102 and/or stored in the database 106. The data in Table 1 can be generated by one or more user devices 104, as previously described. Each entry in Table 1 is labeled with a user ID, and includes a time stamp indicating when the data was obtained, the type of data, and the data value.

TABLE 1
Health and Behavioral Patient Data

| User ID | Time | Data Type | Value | |
|---|---|---|---|---|
| user1 | 2018 08 30 7:48:15.124 utc | blood glucose | 135 | mg/dL |
| user2 | 2018 08 30 7:48:15.126 utc | carbohydrates | 38 | g |
| user3 | 2018 08 30 7:48:16.324 utc | activity | 30 | min |
| user2 | 2018 08 30 7:48:17.128 utc | medicine: insulin | 6 | U |
| user4 | 2018 08 30 7:48:15.226 utc | blood glucose | 218 | mg/dL |
| user1 | 2018 08 30 7:48:15.829 utc | carbohydrates | 14 | g |

| | | | |
|---|---|---|---|
| user5 | 2018 08 30 7:48:17.155 utc | a1c | 7.80% |

As another example, Table 2 below illustrates exemplary personal data that may be provided to the system 102 and/or stored in the database 106. The data in Table 1 can be generated by one or more user devices 104, as previously described. Each entry in Table 2 is labeled with a user ID, and includes personal information for that particular patient such as the time zone in which the patient is located, the type of diabetes the patient has, the date that the patient was first enrolled in the system 102, the year in which the patient was diagnosed with diabetes, and the patient's gender.

TABLE 2
Personal Data

| User | Time | Diabetes | | Diagnosis | |
|---|---|---|---|---|---|
| ID | Zone | Type | Start Date | Year | Gender |
| user1 | New York | Type 2 | 2014 Mar. 5 | 2002 | F |
| user2 | Los Angeles | Type 1 | 2016 Dec. 26 | None | M |
| user3 | Mumbai | Type 2 | 2015 Apr. 8 | 2015 | None |
| user4 | Lisbon | Type 2 | 2017 Sep. 13 | None | M |

In some embodiments, one or more users can access the system 102 via the user devices 104, e.g., to send data to the system 102 (e.g., blood glucose data, other patient data), receive data from the system 102 (e.g., a blood glucose forecast), etc. The users can be individual users (e.g., patients, healthcare professionals, etc.), computing devices, software applications, objects, functions, and/or any other types of users and/or any combination thereof. For example, upon obtaining appropriate data (e.g., blood glucose data, health data, etc. as discussed above), the user device 104 can generate an instruction and/or command to the system 102, e.g., to process the obtained data, store the data in the database 106, extract additional data from one or more databases, and/or perform analysis of the data. The instruction/command can be in a form of a query, a function call, and/or any other type of instruction/command. In some implementations, the instructions/commands can be provided using a microphone (either a separate microphone or a microphone imbedded in the user device 104), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, and/or using any other device. The user device 104 can also instruct the system 102 to perform an analysis of data stored in the database 106 and/or inputted via the user device 104.

As discussed further below, the system 102 can analyze the obtained data, including past data, continuously supplied data, and/or any other data (e.g., using a statistical analysis, machine learning analysis, etc.), and generate a forecast of an expected blood glucose state (e.g., blood glucose level, hypoglycemia event, hyperglycemia event) for the patient. Optionally, the system 102 can also provide interpretations, recommendations, notifications, or other information related to the obtained data and/or the forecasted blood glucose state. The system 102 can perform such analyses at any suitable frequency and/or any suitable number of times (e.g., once, multiple times, on a continuous basis, etc.). For example, when updated data is supplied to the system 102 (e.g., from the user devices 104), the system 102 can reassess and update its previous prediction, if appropriate. In performing its analysis, the system 102 can also generate additional queries to obtain further information (e.g., from the user devices 104, the database 106, or third party sources). In some embodiments, the user device 104 can automatically supply the system 102 with such information. Receipt of updated/additional information can automatically trigger the system 102 to execute a process for reanalyzing, reassessing, or otherwise updating previous predictions.

For example, as described in greater detail below, the system 102 can be supplied with at least one of the following types of input data for executing an analysis: data logged from one or more CGM devices that measure and report a patient's blood glucose levels at a predetermined time interval (e.g., once every 5 to 10 minutes), data indicating the patient's insulin intake (e.g., entered by the patient via the mobile device 104b), data indicating the patient's meal intake (e.g., entered by the patient via the mobile device 104b), and/or data indicating the patient's physical activity (e.g., logged by a wearable device 104c). In other embodiments, however, any other data can be provided to and/or used by the system 102, such as any of the data described herein.

In some embodiments, the system 102 is configured to forecast the patient's blood glucose state using one or more machine learning models. The machine learning models can include supervised learning models, unsupervised learning models, semi-supervised learning models, and/or reinforcement learning models. Examples of machine learning models suitable for use with the present technology include, but are not limited to: regression algorithms (e.g., ordinary least squares regression, linear regression, logistic regression, stepwise regression, multivariate adaptive regression splines, locally estimated scatterplot smoothing), instance-based algorithms (e.g., k-nearest neighbor, learning vector quantization, self-organizing map, locally weighted learning, support vector machines), regularization algorithms (e.g., ridge regression, least absolute shrinkage and selection operator, elastic net, least-angle regression), decision tree algorithms (e.g., classification and regression trees, Iterative Dichotomiser 3 (ID3), C4.5, C5.0, chi-squared automatic interaction detection, decision stump, M5, conditional decision trees), Bayesian algorithms (e.g., naïve Bayes, Gaussian naïve Bayes, multinomial naïve Bayes, averaged one-dependence estimators, Bayesian belief networks, Bayesian networks), clustering algorithms (e.g., k-means, k-medians, expectation maximization, hierarchical clustering), association rule learning algorithms (e.g., apriori algorithm, ECLAT algorithm), artificial neural networks (e.g., perceptron, multilayer perceptrons, back-propagation, stochastic gradient descent, Hopfield networks, radial basis function networks), deep learning algorithms (e.g., convolutional neural networks, recurrent neural networks, long short-term memory networks, stacked auto-encoders, deep Boltzmann machines, deep belief networks), dimensionality reduction algorithms (e.g., principle component analysis, principle component regression, partial least squares regression, Sammon mapping, multidimensional scaling, projection pursuit, discriminant analysis), time series forecasting algorithms (e.g., exponential smoothing, autoregressive models, autoregressive with exogenous input (ARX) models, autoregressive moving average (ARMA) models, autoregressive moving average with exogenous inputs (ARMAX) models, autoregressive integrated moving average (ARIMA) models, autoregressive conditional heteroskedasticity (ARCH) models), and ensemble algorithms (e.g., boosting, bootstrapped aggregation, AdaBoost, blending, stacking, gradient boosting machines, gradient boosted trees, random forest). Additional examples of machine learning models suitable for use with the forecasting techniques herein are discussed further below.

Although FIG. 1 illustrates a single set of user devices 104, it will be appreciated that the system 102 can be operably and communicably coupled to multiple sets of user devices, each set being associated with a particular patient or user. Accordingly, the system 102 can be configured to receive and analyze data from a large number of patients (e.g., at least 50, 100, 200, 500, 1000, 2000, 3000, 4000, 5000, or 10,000 different patients) over an extended time period (e.g., weeks, months, years). The data from these patients can be used to train and/or refine one or more machine learning models implemented by the system 102, as described below.

The system 102 and user devices 104 can be operably and communicatively coupled to each other via the network 108. The network 108 can be or include one or more communications networks, and can include at least one of the following: a wired network, a wireless network, a metropolitan area network (“MAN”), a local area network (“LAN”), a wide area network (“WAN”), a virtual local area network (“VLAN”), an internet, an extranet, an intranet, and/or any other type of network and/or any combination thereof. Additionally, although FIG. 1 illustrates the system 102 as being directly connected to the database 106 without the network 108, in other embodiments the system 102 can be indirectly connected to the database 106 via the network 108. Moreover, in other embodiments one or more of the user devices 104 can be configured to communicate directly with the system 102 and/or database 106, rather than communicating with these components via the network 108.

The various components 102-108 illustrated in FIG. 1 can include any suitable combination of hardware and/or software. In some embodiment, components 102-108 can be disposed on one or more computing devices, such as, server(s), database(s), personal computer(s), laptop(s), cellular telephone(s), smartphone(s), tablet computer(s), and/or any other computing devices and/or any combination thereof. In some embodiments, the components 102-108 can be disposed on a single computing device and/or can be part of a single communications network. Alternatively, the components can be located on distinct and separate computing devices.

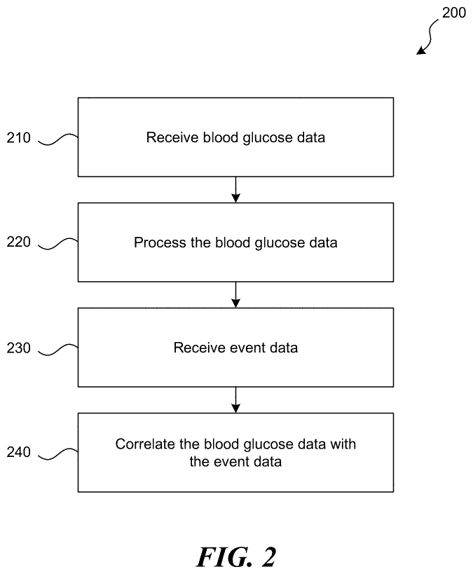

FIG. 2 is a flowchart illustrating a method 200 for preparing blood glucose data for use in biomonitoring and forecasting, in accordance with embodiments of the present technology. The method 200 can be performed by any of the systems and devices described herein. For example, some or all of the steps of the method 200 can be performed by the system 102 and/or the user devices 104 of FIG. 1. In some embodiments, the method 200 is performed by a computing system or device including one or more processors and a memory storing instructions that, when executed by the one or more processors, cause the computing system or device to perform one or more of the steps described herein. The method 200 can be executed in order to augment a patient's blood glucose data with information relevant to the blood glucose analysis and/or forecasting techniques described herein.

The method 200 begins at step 210 with receiving blood glucose data. The blood glucose data can be received from a user device, such as a CGM device or other blood glucose sensor (e.g., blood glucose sensor 104a of FIG. 1). As previously described, the blood glucose data may be CGM data including a plurality of blood glucose measurements taken at a relatively particular time interval (e.g., once every 5-10 minutes). The blood glucose measurements can be taken over any suitable time period, e.g., over 15 minutes, 30 minutes, 45 minutes, 1 hour, 2 hours, 5 hours, 10 hours, 12 hours, 24 hours, 36 hours, or 48 hours, or longer.

At step 220, the blood glucose data is processed. The processing can include, for example, partitioning the data into one or more substantially uninterrupted series of blood glucose measurements, also referred to herein as “episodes.” A series of blood glucose measurements may be considered to be substantially uninterrupted if, for example, the number, size, and/or frequency of gaps in the measurements is sufficiently small (e.g., below a predetermined threshold). For example, a substantially uninterrupted series of measurements may not include any gaps that are greater than or equal to 2× the normal time interval between readings (e.g., if measurements are normally taken every 5 minutes, there are no gaps between measurements that are 10 minutes or longer).

Step 220 can also include discarding episodes that are shorter than a predetermined minimum time period, e.g., due to potential reliability issues. The minimum time period can be, for example, 15 minutes, 30 minutes, 45 minutes, 60 minutes, 90 minutes, or 2 hours. In some embodiments, step 220 further includes smoothing the blood glucose data, e.g., to reduce volatility, remove noise, and/or remove erroneous data. The smoothing can be performed using filtering algorithms or any other suitable algorithms known to those of skill in the art.

At step 230, event data is received. Event data can include any data other than blood glucose data that may be relevant to the patient's blood glucose state. The event data can be associated with a health-related event experienced by the patient at a particular time point and/or over a particular time period. Accordingly, the event data can include data regarding the timing of the event (e.g., time stamps, duration), as well as other data indicative of event parameters that may influence the patient's blood glucose level. For example, event data can include insulin intake data (e.g., basal and/or bolus dosage), food intake (e.g., type of food, calories consumed, carbohydrates consumed), and/or physical activity data (e.g., type of activity, duration of activity, activity level, calories burned). Event data can also include data of other physiological parameters and/or biological markers, such as blood pressure data, sleep data, heart rate data, skin temperature data, data of chemical indictors of stress level (e.g., cortisol) or other conditions, etc.

In some embodiments, the event data is received by a device (e.g., the mobile device 104b, wearable device 104c, and/or any other user devices 104 of FIG. 1) that is operated by or otherwise associated with the patient. The device can be the same device used to generate the blood glucose data, or can be a different device. The event data can be generated automatically by the device and/or can be manually input into the device by the patient. The event data can be received before, after, and/or concurrently with the blood glucose data.

At step 240, the blood glucose data is correlated with the event data. Step 240 can include, for example, combining and/or annotating the blood glucose data with the event data so that the timing of the event data can be determined with reference to the timing of the blood glucose data. In some embodiments, the blood glucose data and event data are organized in order of timing and combined into a single data structure (e.g., a data table or matrix). Blood glucose data that has been correlated with event data (also referred to herein as “augmented episodes”) can then be stored and/or used in the analysis and forecasting techniques described herein.

In some embodiments, one or more correlations between the event data and blood glucose data can be identified. The blood glucose data can be annotated based on the correlations. Subsets of event data and blood glucose data can be used. For example, event data associated with blood glucose level changes above a threshold can be in a first data structure, and event data associated with blood glucose level changes below the threshold can be in a second data structure. Event data can also be grouped based on, for example, duration characteristics (e.g., events that affect blood glucose for predetermined periods of time), characteristics of blood glucose levels (e.g., events causing rapid changes to blood glucose levels), or the like.

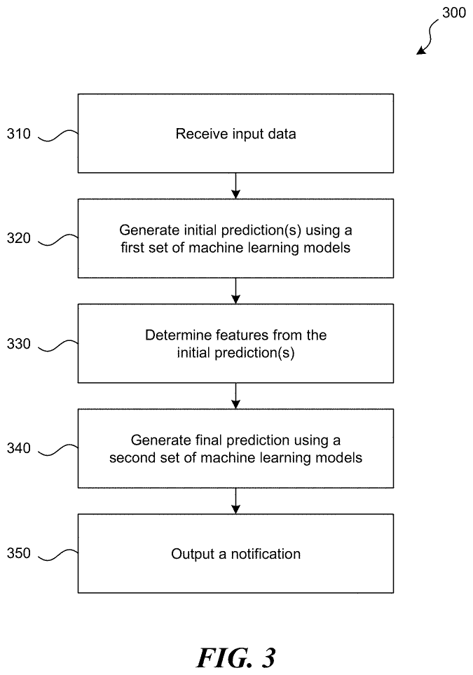

FIG. 3 is a block diagram illustrating a method 300 for forecasting a blood glucose state of a patient, in accordance with embodiments of the present technology. The method 300 can be used to predict a blood glucose state of the patient, such as a blood glucose level or concentration, an occurrence of a hypoglycemia event, and/or an occurrence of a hyperglycemia event. The prediction can be for a future time point (e.g., a blood glucose state at a time point that is 15 minutes, 30 minutes, 60 minutes, 90 minutes, 2 hours, or 4 hours into the future), or for a future time period (e.g., a blood glucose state over the next 15 minutes, 30 minutes, 60 minutes, 90 minutes, 2 hours, 4 hours, or overnight). For example, the method 300 can be used to predict one or more blood glucose values at one or more future time points, such as the forecasted blood glucose level at a certain time interval (e.g., every 2 minutes, 5 minutes, 10 minutes, 15 minutes) over a specified time period (e.g., the next 30 minutes, 60 minutes, 90 minutes, 2 hours, 4 hours, or overnight). As another example, the method 300 can be used to predict, for a particular future time period, whether the patient's blood glucose levels are likely to fall below a particular threshold value (e.g., a threshold for hypoglycemia), whether the patient's blood glucose levels are likely to rise above a particular threshold value (e.g., a threshold for hyperglycemia), whether a hypoglycemia event is likely to occur (e.g., in terms of low, medium, or high risk), whether a hyperglycemia event is likely to occur (e.g., in terms of low, medium, or high risk), and so on.

The method 300 begins at step 310 with receiving input data. The input data can include any suitable data of the patient as described herein, such as blood glucose data (e.g., continuous blood glucose data generated by a CGM device), insulin intake data, food intake data, physical activity data, etc. In some embodiments, the input data includes one or more episodes of blood glucose data, which may be processed (e.g., smoothed) and/or correlated with at least event as previously described with respect to the method 200 of FIG. 2. Optionally, the input data can include only a single episode of blood glucose data (e.g., the most recent episode of the patient), which can be annotated or otherwise correlated with one or more events (e.g., insulin intake events, food intake events, physical activity events, etc.). The data may be obtained from various sources, e.g., inputted by the user, queried from one or more databases, obtained from CGM sensors or other user devices, etc.

In some embodiments, the input data also includes averages, standard deviations, maxima, minima and/or other statistics calculated from the patient's historical blood glucose levels and/or other historical data of the patient (e.g., historical event data). These statistics can be calculated to determine trends, patterns, etc. in the patient's glucose levels and/or other activities or parameters at a particular time of day, which can be useful when making predictions for a particular time point or time period. For example, in embodiments where a blood glucose level prediction is being made for a particular hour of the day (e.g., from 4 PM to 5 PM), the input data can also include an average and/or standard deviation of the patient's blood glucose level for that time of day, computed based on the patient's previously recorded blood glucose data (e.g., all previous blood glucose data up to the current day).

At step 320, at least one initial prediction is generated using a first set of machine learning models. Specifically, the input data (e.g., an augmented episode) is input into the first set of machine learning models, and the first set of machine learning models use the input data to generate the initial prediction(s). The first set of machine learning models can include any suitable number of machine learning models, such as one, two, three, four, or more different machine learning models. In embodiments where the first set includes multiple machine learning models, each model can independently generate a respective initial prediction of the patient's blood glucose state. For example, depending on the number of machine learning models in the first set, step 320 can include generating one, two, three, four, or more initial predictions. Optionally, some or all of the outputs of the machine learning models can be combined with each other to generate the initial prediction (e.g., using weighted averages, etc.).

The first set of machine learning models can include any suitable type of machine learning model, such as one or more of the machine learning models previously described with respect to FIG. 1. Each machine learning model can be trained on a respective set of training data. In embodiments where the first set of machine learning models includes multiple machine learning models, some or all of the models can be trained on the same training data, or some or all of the models can be trained on different training data. The training data can include, for example, previous data from the same patient, such as previous blood glucose data (e.g., episodes prior to the current episode), previous insulin intake data, previous food intake data, previous physical activity data, personal data, physiological data, and/or any other type of data described herein. Alternatively or in combination, the training data can include data from other patients, such as blood glucose data, insulin intake data, food intake data, physical activity data, personal data, physiological data, and/or any other suitable data from a plurality of different patients. In some embodiments, the training data is or includes episodes of blood glucose data that have been correlated and/or annotated with one or more events, as previously described with respect to the method 200 of FIG. 2.

The initial prediction(s) generated by the first set of machine learning models can be a prediction of one or more future blood glucose levels, a hypoglycemia event, a hyperglycemia event, or a combination thereof. For example, the initial prediction(s) can include a time series of blood glucose values at a specified time interval over a specified time period (e.g., every 5 minutes for the next 1-2 hours). The initial prediction(s) can optionally include a calculated confidence interval or other indicator of uncertainty for each predicted blood glucose value. In embodiments where the first set of machine learning models includes multiple different machine learning models, each model can produce a respective time series of blood glucose predictions. Optionally, the initial prediction(s) can be filtered, e.g., to exclude predictions that are outliers, inconsistent with the input data, and/or contradictory. Filtering can also be performed to exclude predictions that are more likely to be inaccurate (e.g., low confidence predictions) while retaining predictions that are more likely to be accurate (e.g., high confidence predictions). Filtering may be applied using various parameters, such as average range of blood glucose levels, physical activity values (e.g., time), carbohydrate consumption (e.g., time, amount, etc.), derivatives of blood glucose levels, maximum and/or minimum blood glucose levels, standard deviation of blood glucose levels, heart rate values, etc. The filtering can be based on values of the filtering parameters in the time period preceding the time period for the prediction (e.g., 30 minutes, 60 minutes, 90 minutes, 2 hours, or 4 hours before the prediction time period).

At step 330, one or more features are determined from the initial prediction(s). The features can include transformations, combinations, statistics, or any other properties or characteristics of the initial prediction(s). Features can include, but are not limited to: averages over a specified time period, standard deviations over a specified time period, trends, fits (e.g., polynomial fits), timing-related features (e.g., duration of events, time elapsed between events), whether certain conditions are true or false (e.g., whether a particular event has occurred), and the like. For example, in embodiments where the initial prediction includes a time series of predicted blood glucose levels, the features extracted from the prediction may include one or more of the following: average blood glucose level, maximum blood glucose level, minimum blood glucose level, standard deviation of the blood glucose level, an amount of time that the patient's blood glucose levels are hyperglycemic or hypoglycemic (e.g., in absolute or relative terms), etc.

Optionally, step 330 can also include generating features from other data, such as the input data from step 310 (e.g., one or more augmented episodes of the patient). Features can also be generated from other data of the patient such as personal data (e.g., age, gender, demographics, diabetes type), previous blood glucose data, meal data, medical history data, exercise data, personal data, medication data, physiological data, or any other data type described herein. Features may be generated from the data using transformations, combinations, statistics, and/or any other suitable technique for determining properties or characteristics of the patient data.

In some embodiments, features may be generated by transforming and aggregating patient data into structured matrices. The transformations that may be used may depend on the type of data, as discussed below. For example, static personal data, such as gender, age, location, diabetes type, etc. may be converted into unordered categorical values. As another example, the features can include at least one of the following: average and/or standard-deviation blood glucose levels, a fraction of time the patient experiences or experienced hyperglycemia and/or hypoglycemia, an average number of nights the patient experienced an overnight hypoglycemia event (e.g., in the past 30 days or other time period), an average physical activity per hour for the patient (e.g., in the past 30 days or other time period), an average and/or standard deviation of blood glucose for the specific hour-of-day (e.g., as known at that time), an average amount of insulin per hour taken by the patient, an average daily insulin intake, an average amount of carbohydrates per hour consumed by the patient, an average maximal range of blood glucose observed within predetermined time periods (e.g., 1 hour, 6 hours, etc.), an average systolic and/or diastolic blood pressure (e.g., in the past 30 days or other time period), an average heart rate, and/or any other data, and/or any combination thereof. The features can include time-related parameters for the time period of the prediction, such as seasonal/cyclical information that may be used as categorical data, such as, for example, but not limited to, day and/or year, hour of day, and/or day-of-week, and/or workday calendar information for the patient's location. Moreover, time-stamped features may include blood glucose values, reported insulin intake, carbohydrates intake, physical activity, al c measurements, weight measurements, and/or any other features and/or any combinations thereof. For blood glucose, the last value, mean, standard deviation, quartiles, and changes over the last observations may be determined over various predefined time periods. For the other inputs, the last, mean, and maximum values may be determined over various predefined time periods.

At step 340, at least one final prediction is generated using a second set of machine learning models. Specifically, the features determined at step 330 are input into the second set of machine learning models, which generates the final prediction. In some embodiments, the features from step 330 are the only input into the second set of machine learning models. In other embodiments, the second set of machine learning models can also receive other inputs, such as the input data of step 310 (e.g., one or more augmented episodes), the initial prediction(s) generated in step 320, and/or other data of the patient (e.g., personal data, previous blood glucose data, meal data, medical history data, exercise data, personal data, medication data, physiological data, etc.).

The second set of machine learning models can be different from the first set of machine learning models. In some embodiments, the second set of machine learning models includes only a single machine learning model. In other embodiments, the second set of machine learning models can include multiple machine learning models whose outputs are combined (e.g., by weighted averages, etc.) to generate a single final prediction. The second set of machine learning models can include any suitable type of machine learning model, such as one or more of the machine learning models previously described with respect to FIG. 1. Each machine learning model can be trained on a respective set of training data. In embodiments where the second set of machine learning models includes multiple machine learning models, some or all of the models can be trained on the same training data, or some or all of the models can be trained on different training data. The training data can include, for example, previous data from the patient, such as previous blood glucose data (e.g., episodes prior to the current episode), previous insulin intake data, previous food intake data, previous physical activity data, and/or any other type of data described herein. Alternatively or in combination, the training data can include data from other patients, such as blood glucose data, insulin intake data, food intake data, physical activity data, and/or any other data from a plurality of different patients. In some embodiments, the training data is or includes blood glucose data that has been annotated with one or more events, as discussed above.

In some embodiments, the training data for the second set of machine learning models includes features generated from data of the patient and/or data of a plurality of other patients. The features can include any of the features previously described with respect to step 330. In some embodiments, for example, the features can be generated from a plurality of patient data sets, each patient data set including personal data (e.g., diabetes type), blood glucose data (e.g., previous and/or current episodes), insulin intake data, food intake data, physical activity data, and/or any other data. Each patient data set can also include blood glucose predictions for the patient that are generated using machine learning models (e.g., the first set of machine learning models). The blood glucose predictions can be retrospective predictions generated from previous blood glucose data. The features generated from these predictions can also be used to train the second set of machine learning models.

The final prediction produced by the second set of machine learning models can be a prediction of one or more future blood glucose levels, a hypoglycemia event, a hyperglycemia event, or a combination thereof. For example, the final prediction can be a predicted series of blood glucose values over a specified time period and at a specified time interval (e.g., every 5 minutes for the next 1-2 hours). As another example, the final prediction can be an estimated likelihood that the patient will experience a hypoglycemia or hyperglycemia event within a specified time period (e.g., the next 15 minutes, 30 minutes, 60 minutes, 90 minutes, 2 hours, 4 hours, or overnight). The likelihood of the hypoglycemia or hyperglycemia event can be expressed in various ways, such as in qualitative terms (e.g., “likely to occur” versus “not likely to occur,” “high risk” versus “moderate risk” versus “low risk”) and/or in quantitative terms (e.g., a probability value). Optionally, the final prediction can be filtered, e.g., to exclude predicted values that are outliers, inconsistent with the input data, and/or contradictory (e.g., as previously described with respect to step 320).

At step 350, the method 300 optionally includes outputting a notification to the patient. The notification can be output by the system for display on a user device (e.g., user devices 104 of FIG. 1) via a graphical user interface, as described in greater detail below. The notification can include information regarding the final prediction of the blood glucose state (e.g., the predicted blood glucose level, the predicted likelihood of hypoglycemia or hyperglycemia, etc.). In some embodiments, the notification includes recommendations or feedback on actions that the patient may take in response to the predicted blood glucose state, e.g., to control the blood glucose level, avoid hyperglycemia or hypoglycemia, etc. For example, the notification may instruct the patient to take medication, consume a meal, exercise, contact a healthcare professional, and so on. Optionally, the notification can be transmitted to a physician or other healthcare professional associated with the patient, e.g., if the final prediction indicates that the patient may need immediate medical attention, if the patient's blood glucose level is consistently too high or too low, or if there are any other situations where the physician should be notified.

The method 300 can be performed by any of the systems and devices described herein, such as a computing system or device including one or more processors and a memory storing instructions that, when executed by the one or more processors, cause the computing system or device to perform one or more of the steps described herein. For example, some or all of the steps of the method 300 can be performed by the system 102 and/or the user devices 104 of FIG. 1. In some embodiments, the process of generating the initial and final predictions uses less computing power and resources than the process of training the first and second sets of machine learning models. For example, the predictions can be generated using a relatively small amount of data (e.g., the patient's current blood glucose data and pre-computed parameters for the first and second sets of machine learning models), while training may involve very large amounts of data (e.g., data from large numbers of patients). Accordingly, the training can be performed on the system 102, while the predictions can be made at a remote location (e.g., via cloud computing) and/or locally on the user devices 104 and/or any other device. In other embodiments, however, the training and forecasting can be performed entirely on the system 102, entirely on a user device 104, or entirely on another computing system or device.

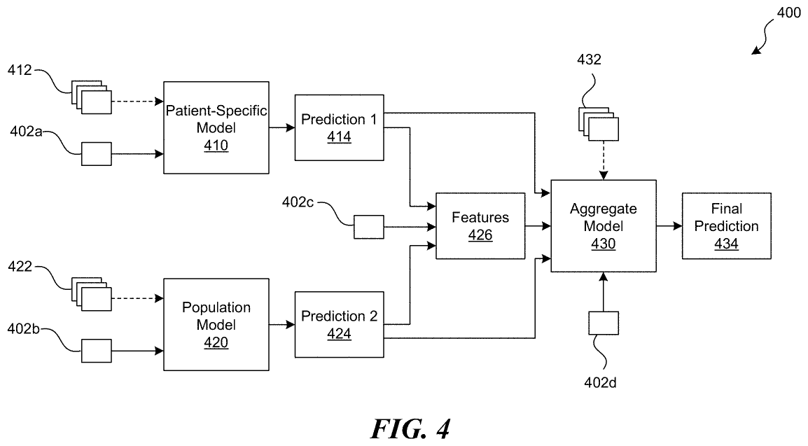

FIG. 4 is a block diagram schematically illustrating a machine learning architecture (“architecture 400”) for blood glucose forecasting configured in accordance with embodiments of the present technology. The architecture 400 can be implemented across software and/or hardware components by any of the systems and devices described herein, such as the system 102 and/or user devices 104 of FIG. 1. The architecture 400 includes three different machine learning models: an individualized or patient-specific model 410, a population model 420, and an aggregate model 430. In some embodiments, the architecture 400 is used to perform a method for forecasting a blood glucose state of a patient, such as the method 300 of FIG. 3. In such embodiments, the patient-specific model 410 and population model 420 may correspond to the first set of machine learning models of the method 300, while the aggregate model 430 may correspond to the second set of machine learning models of the method 300.

The patient-specific model 410 can be a machine learning model that is trained on data of the particular patient for which a prediction is to be made (“patient-specific training data 412”). The patient-specific training data 412 may only include data from a single patient. In some embodiments, the patient-specific training data 412 includes a plurality of blood glucose episodes that are correlated and/or annotated with event data (e.g., insulin intake events, food intake events, physical activity events, etc.), as previously described with respect to FIG. 2. Optionally, the patient-specific training data 412 can include other types of data (e.g., any of the data described above with respect to FIG. 1).

The patient-specific model 410 can be or include any suitable type of machine learning model, such as a time-series forecasting model or a combination of time-series models. For example, the patient-specific model 410 can be or include an ARIMA model. By way of a non-limiting example, the ARIMA model may be expressed as follows:

(1−Σᵢ₌₁^{p}φᵢLⁱ)(1−L)^{d}Xₜ=δ+(1+Σᵢ₌₁^{q}θᵢLⁱ)εₜ (1)

where φᵢ and θᵢ are scalar elements in vectors, p, q, and d are scalars, εₜ are error terms, and L is a lag operator that backshifts an element x in a series such that L^{k}xₜ=xₜ₋ₖ. At every time point, the vectors φᵢ and θᵢ may be fitted to minimize errors in the observed series, while the scalars, p, q, and d may be selected by estimating the Akaike information criterion (AIC) for each triplet in the search space and selecting one which produces the minimal error value. In some embodiments, the ARIMA model is modified to accept exogenous events (e.g., insulin intake events, food intake events, physical activity events, etc.) as well as time series blood glucose data.

The population model 420 can be a machine learning model that is trained on data from a plurality of patients (“population training data 422”). For example, the population training data 422 can include data from at least 50, 100, 200, 500, 1000, 2000, 3000, 4000, 5000, or 10,000 different patients. Optionally, the population training data 422 can also including data from the particular patient for which a prediction is to be made. Each patient data set can include a plurality of blood glucose episodes that are correlated and/or annotated with event data, as previously described. In some embodiments, the population training data 422 includes at least 100,000 hours, 500,000 hours, 1 million hours, 5 million hours, or 10 million hours of episodes combined across the plurality of patients. In some embodiments, the population training data 422 can include population data from a group of patients selected based on condition (e.g., Type 1 diabetes, Type 2 diabetes, and gestational diabetes), age, gender, race, demographics, etc. For example, the selected patients can have characteristics similar to the patient for which the prediction is being made (e.g., in terms of diabetes type, age, gender, race, demographics, etc.). Optionally, the population training data 422 can also include other types of data (e.g., any of the data described above with respect to FIG. 1).

The population model 420 can be or include any suitable type of machine learning model, such a deep learning model. In some embodiments, for example, the population model 420 is or includes a deep learning autoregressive recurrent neural network model. The machine learning model used for the population model 420 can be different from the machine learning model used for the patient-specific model 410. In other embodiments, however, the patient-specific model 410 and population model 420 can use the same machine learning model. In some embodiments, the population model 420 is selected for the particular patient. For example, the population training data 422 and population model 420 can be selected based on one or more criteria, such as the patient's condition (e.g., diabetes type), age, gender, demographics, race, etc. In other embodiments, the same population training data 422 and population model 420 can be used with all of the patients.

The aggregate model 430 can be a machine learning model that is trained on feature data generated from a plurality of patient data sets (“aggregate training data 432”). The feature data can include a plurality of features (e.g., transformations, combinations, statistics, properties, characteristics, etc.) generated from the patient data sets. The patient data sets can include data from at least 50, 100, 200, 500, 1000, 2000, 3000, 4000, 5000, or 10,000 different patients. The patient data sets can also include data from the patient for which a prediction is to be made. Each patient data set can include a plurality of blood glucose episodes that are annotated with event data, as previously described. In some embodiments, the patient data sets include at least 100,000 hours, 500,000 hours, 1 million hours, 5 million hours, or 10 million hours of episodes combined across the plurality of patients. In some embodiments, each patient data set includes other data for that particular patient, such as a personal data (e.g., age, gender, demographics, diabetes type), meal data, medical history data, exercise data, medication data, physiological data, or any other data type described herein.

In some embodiments, each patient data set also includes blood glucose predictions that are generated using machine learning models. For example, each patient data set can include a set of predictions generated by an individualized model trained on data for that particular patient (e.g., similar to the patient-specific model 410), and a set of predictions generated by a population model trained on data from a plurality of patients (e.g., the population model 420). The blood glucose predictions can be retrospective predictions generated from previous blood glucose data of that patient. The features generated from these predictions can also be included in the feature data used to train the aggregate model 430. In some embodiments, the predictions that are used to generate features vary depending on the time horizon of the predictions to be made by the aggregate model 430. For example, a model that is used to make 1 hour predictions can be trained on features extracted from 1 hour predictions, a model that is used to make 2 hour predictions can be trained on features from 2 hour predictions, and so on.

In some embodiments, the aggregate training data 432 can also include the patient data sets, in addition to the feature data computed from those patient data sets. In other embodiments, the aggregate training data 432 may only include feature data, such that the patient data sets are not directly used to train the aggregate model 430.

By proceeding as described above with features computed from the historical data of many patients, a large volume of examples of individual predictions, processed data from those individuals, and actual blood glucose value(s) (e.g., as reported by the patients' CGM devices) may be generated. The volume of examples may be used to train the aggregate model 430 to predict blood glucose concentration (e.g., using supervised learning). The aggregate model 430 may synthesize a large amount of data and predictions for individual patients based on a broader set of examples from many other individual patients to generate more accurate predictions of blood glucose levels.

The aggregate model 430 can be or include any suitable type of machine learning model, such as a decision trees model. In some embodiments, for example, the aggregate model 430 is a gradient boosted trees model. The machine learning model used for the aggregate model 430 can be different from the machine learning models used for the patient-specific model 410 and/or the population model 420. In other embodiments, however, the aggregate model 430 can use the same machine learning model as the patient-specific model 410 and/or the population model 420.

In some embodiments, the machine learning model used for the aggregate model 430 can be selected based on the particular type of prediction to be made (e.g., blood glucose level, hypoglycemia prediction, hyperglycemia prediction) and/or the timing for the prediction (e.g., 30 minute forecast, 1 hour forecast, 2 hour forecast, overnight forecast, etc.). For example, in embodiments where the final prediction is a prediction of a blood glucose level or time series of blood glucose levels, the aggregate model 430 can a regression algorithm that forecasts a specific value or values. A regression algorithm can be configured to map a set of features to a particular objective with the aim to minimize some predefined loss related to that objective. The objective for which the regression algorithm optimizes may be selected either as the future blood glucose level at a particular time, and/or a minimal (and/or maximal) blood glucose level to occur within some time period in the future, based on the target to be predicted. As another example, in embodiments where the final prediction is a prediction of a hypoglycemia or hyperglycemia event, the aggregate model 430 can be a classification algorithm that forecasts a label or state (e.g., true or false, risk level). A classification model can be configured to learn a mapping from a set of input points to a target class or category.

The patient-specific model 410, population model 420, and/or aggregate model 430 can be periodically updated as new patient data is received. The models 410-430 can be updated at the same frequency, or can be updated at different frequencies (e.g., depending on the complexity of the model, the size of the training data set, the time to update the model, etc.). In some embodiments, the patient-specific model 410 is updated at a higher frequency (e.g., once per day), the population model 420 is updated at a high or intermediate frequency (e.g., once per day, once per week), and the aggregate model 430 is updated at a lower frequency (e.g., once per month, once per quarter).

To forecast a patient's future blood glucose state, blood glucose data from the patient (e.g., “patient data”) is input into the patient-specific model 410 and the population model 420. Specifically, first patient data 402a is input into the patient-specific model 410 and second patient data 402b is input into the population model 420. As discussed above, the first and second patient data 402a, 402b can each include one or more blood glucose episodes along with event data, such as the most recent episode experienced by the patient. The first and second patient data 402a, 402b can each be obtained using a CGM device and/or any other user device (e.g., user devices 104 of FIG. 1), as previously described. The first patient data 402a may be the same as the second patient data 402b, or may be different from the second patient data 402b. In some embodiments, for example, the first patient data 402a also includes average blood glucose levels and/or standard deviations of blood glucose levels for the particular time of day for which the prediction is being made (e.g., the patient's average blood glucose value from 4 PM to 5 PM each day). These averages and/or standard deviations can be computed based on previous blood glucose data of the patient (e.g., all historical blood glucose data up to the current day). The second patient data 402b may or may not include these average and/or standard deviation values.

The patient-specific model 410 can generate a first prediction 414 and the population model 420 can generate a second prediction 424. For example, the first prediction 414 can be a first time series of blood glucose values (e.g., every 5 minutes for the next 1-2 hours) and the second prediction 424 can be a second time series of blood glucose values (e.g., every 5 minutes for the next 1-2 hours). The first prediction 414 and second prediction 424 are used to generate features 426. Optionally, the features 426 can also be generated based at least in part on third patient data 402c (e.g., one or more blood glucose episodes with event data) and/or other data (e.g., personal data such as diabetes type). The third patient data 402c may be the same as the first patient data 402a and the second patient data 402b. In other embodiments, the third patient data 402c may be different from the first patient data 402a and/or the second patient data 402b.

The features 426 can be input into the aggregate model 430. Optionally, the first prediction 414, second prediction 424, and/or fourth patient data 402d (e.g., one or more blood glucose episodes with event data) can also be inputs for the aggregate model 430. The fourth patient data 402d may be the same as the first patient data 402a, the second patient data 402b, and the third patient data 402c. In other embodiments, the fourth patient data 402d may be different from the first patient data 402a, the second patient data 402b, and/or the third patient data 402c.

As discussed above, the specific machine learning model for the aggregate model 430 can be selected based on the type of prediction to be made (e.g., 1-hour blood glucose level forecast, 2-hour blood glucose level forecast, 1-hour hypoglycemia prediction, 2-hour hypoglycemia prediction, etc.). The aggregate model 430 can generate a final prediction 434, which can be a prediction of one or more future blood glucose levels (e.g., a time series of blood glucose values over a future time period), a predicted likelihood of a hypoglycemia event, a predicted likelihood of a hyperglycemia event, or a combination thereof.

In some embodiments, the architecture 400 is used to generate predictions for a patient whose earlier data was included in the population training data 422 and the aggregate training data 432 (also referred to herein as a “seen user”). In other embodiments, however, the architecture 400 can be used to generate predictions for a patient whose data was not included in the population training data 422 and/or the aggregate training data 432 (also referred to herein as an “unseen user”). To do so, patient-specific input data may be determined and input into the patient-specific model 410 and/or the population model 420 to generate initial predictions for the particular patient. The prediction results may be used to compute features that are input into the aggregate model 430, as discussed above. In some embodiments, the accuracies of blood glucose predictions for unseen users may be similar to the accuracies of blood glucose predictions for seen users.

Although FIG. 4 illustrates a particular configuration for the architecture 400, it will be appreciated that the architecture 400 can be configured in many different ways. For example, although FIG. 4 shows the aggregate model 430 receiving the features 426, first prediction 414, second prediction 424, and fourth patient data 402d as inputs, in other embodiments, one or more of these inputs can be omitted. Likewise, although FIG. 4 shows the features 426 being generated from the first prediction 414, the second prediction 424, and the third patient data 402c, in other embodiments, one or more of these inputs can be omitted. As another example, features extracted from the first patient data 402a can be used as inputs for the patient-specific model 410, in combination with or as an alternative to the patient data 402a itself. Similarly, features extracted from the second patient data 402b can be used as inputs for the population model 420, in combination with or as an alternative to the patient data 402b itself. In yet another example, the patient-specific model 410 or the population model 420 may be omitted. Optionally, in other embodiments, the aggregate model 430 can include multiple machine learning models that are blended, stacked, or otherwise combined with each other.

### Overnight Hypoglycemia Prediction

Hypoglycemia events can occur overnight and may be particularly dangerous for individuals with diabetes. Properly and timely administered care for such hypoglycemic events is essential. However, many individuals suffering from diabetes and experiencing a hypoglycemic event overnight may be unable to perform the necessary steps to address this condition. This may be due to grogginess from being awakened at night that may compound the confusion and clumsiness already associated with low blood sugar, making it more challenging to administer adequate self-care. Conventional systems may not be capable of providing the necessary tools to allow diabetic patients to take appropriate steps, e.g., prior to going to bed, to prevent overnight hypoglycemia when they suspect such an episode may occur while they are sleeping. Thus, there is a need to forecast a probability of experiencing an overnight hypoglycemic event and provide tailored recommendations to inform users of the most effective actions that they can take to prevent such events.

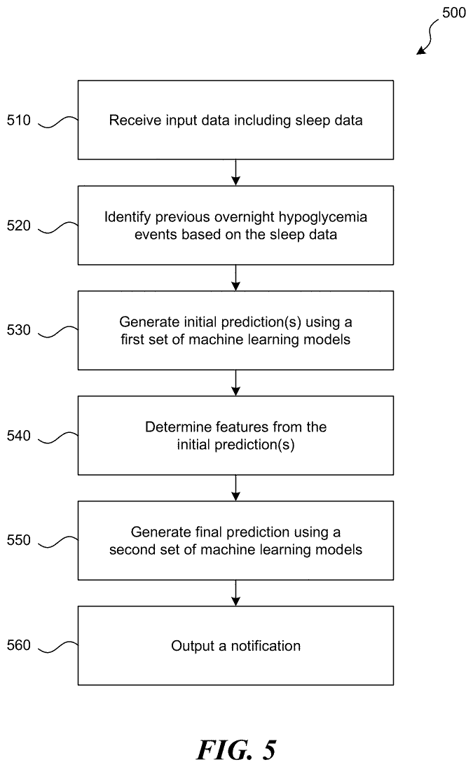

FIG. 5 is a block diagram illustrating a method 500 for forecasting an overnight hypoglycemia event, in accordance with embodiments of the present technology. The method 500 can be performed using any of the systems and devices described herein (e.g., system 102 and/or user devices 104 of FIG. 1). The method 500 can be generally similar to the method 300 described above with respect to FIG. 3. Accordingly, the discussion of the method 500 will be limited to those elements that differ from the method 300.

The method 500 begins at step 510 with receiving input data including sleep data. Step 510 can be generally similar to step 310 of the method 300, except that the input data also includes sleep data of the patient, which can include the timing of sleep (e.g., when the patient goes to sleep (“bedtimes”), when the patient wakes up), number of hours of sleep, average hours of sleep, variability of hours of sleep, sleep-wake cycle data, data related to sleep apnea events (if any), sleep fragmentation (e.g., fraction of nighttime hours awake between sleep episodes, etc.), frequency of low blood glucose concentration (e.g., <70 mg/dL) while the patient is sleeping, etc. during one or more previous nights. Sleep data can be provided automatically, (e.g., via sleep trackers), manually (e.g., by user input into a mobile device or other user device), or by any other suitable technique and/or device.

At step 520, previous overnight hypoglycemia events are identified based on the sleep data. To do so, for example, average bedtimes and nighttime durations from the patient's sleep data may be ascertained. The sleep data may be analyzed to determine start and end times of sleep periods. The sleep periods may be aggregated (e.g., starting at 7 PM each day). In some embodiments, every aggregated set of sleep periods that lasts between 3 and 9 hours may be considered for the purposes of the model. For patients that have no associated sleep data, a fixed bedtime (e.g., 11 PM) and nighttime duration (e.g., 7 hours) values may be considered. The patient's blood glucose data for each night can then be analyzed to identify and record minimum blood glucose concentration values. If the minimum blood glucose concentration value is lower than a threshold value (e.g., 70 mg/dL), it may be considered as an overnight hyperglycemia event. As can be understood, any threshold value may be used.

At step 530, at least one initial prediction is generated using a first set of machine learning models, as previously described with respect to step 320 of the method 300. In some embodiments, the first set of machine learning models can also be trained on sleep data and/or data of the previous overnight hypoglycemia events (e.g., of the particular patient and/or of a larger patient population). For example, a patient-specific model (e.g., patient-specific model 410 of FIG. 4) can be trained on sleep data and/or overnight hypoglycemia data of the patient, while a population model (e.g., population model 420 of FIG. 4) can be trained on sleep data and/or overnight hypoglycemia data of a plurality of patients. The initial prediction(s) produced by the first set of machine learning models can provide probabilistic estimations of the blood glucose values that may be aggregated to form a probability distribution. In some embodiments, the initial prediction(s) include a series of predicted blood glucose values over a time period where the patient is expected to be asleep (e.g., overnight).

At step 540, one or more features are determined from the initial prediction(s). Step 540 can be generally similar to step 330 of the method 300, except that features can also be generated based on the sleep data and/or overnight hypoglycemia data from steps 510 and 520, respectively. Such features may include, for example, average and/or standard-deviation blood glucose levels (e.g., before bedtime, while the patient is sleeping), a fraction of time the patient experiences or experienced hypoglycemia while sleeping, an average number of nights the patient experienced an overnight hypoglycemia event (e.g., in the past 30 days or other time period), an amount of physical activity before bedtime, an average amount of insulin intake before bedtime, an average amount of carbohydrates consumed before bedtime, an average systolic and/or diastolic blood pressure (e.g., before bedtime, while sleeping), an average heart rate (e.g., before bedtime, while sleeping), parameters for a quadratic fit of the recent blood glucose values before bedtime (e.g., intercept, first order coefficient, second order coefficient), probability of experiencing hypoglycemia up to that day, and/or any other data, and/or any combination thereof.

At step 550, at least one final prediction is generated using a second set of machine learning models, as previously described with respect to step 340 of the method 300. In some embodiments, the second set of machine learning models (e.g., aggregate model 430 of FIG. 4) can also be trained on features extracted from sleep data and/or data of the previous overnight hypoglycemia events (e.g., of the particular patient and/or of a larger patient population). The features can include any of the features described above in step 540. The second set of machine learning models can also be trained on features extracted from predictions of overnight hypoglycemia events (e.g., retrospective predictions generated from previous patient data). The second set of machine learning models may be configured to synthesize the large amount of data and/or predictions for individual patients based on the broader set of examples from many patients to achieve more accurate predictions of the probability of an overnight hypoglycemic event.

The second set of machine learning models can generate a set of predicted probabilities that the patient will experience hyperglycemia during the next overnight period. Optionally, filtering can be applied to the generated probability predictions. In particular, the filtering may be applied to exclude various predictions that are outliers, and/or are inconsistent with the user data, and/or contradictory. Filtering may be applied using various parameters, which may include at least one of the following: average range of blood glucose levels, physical activity values (e.g., time), carbohydrate consumption (e.g., time, amount, etc.), derivatives of blood glucose levels, maximum and/or minimum blood glucose levels, standard deviation of blood glucose levels, heart rate values, etc.

In some embodiments, probability predictions may be used as classifiers and their accuracy may be described by the area under the receiver operating characteristic curve (“ROC AUC”). Optionally, prediction accuracies may be improved by applying one or more filtering parameters to discard predictions that do not meet at least one criterion. Some exemplary filtering criteria may include (as can be understood any other criteria and/or values may be used):
average range of blood glucose levels in the 6 hours preceding bedtime 70 mg/dL;
activity 1 hour prior to bedtime 10 minutes;
activity 6 hours prior to bedtime 40 minutes;
carbohydrate consumption 6 hours prior to bedtime 40 g carbohydrates;
second derivative of blood glucose levels over the 1 hour prior to bedtime between −0.008 and 0.008 mg/dL/min/min;
maximum blood glucose concentration values in the last 2 hours 100 mg/dL;
minimum blood glucose concentration values in the last 2 hours 70 mg/dL;
user's blood glucose concentration values standard deviation 40 mg/dL; and/or
heart rate between 60 and 80 bpm.

At step 560, the method 500 optionally includes outputting a notification to the patient, as described above with respect to step 350 of the method 300. In some embodiments, the notification includes the calculated probability of overnight hypoglycemia, e.g., displayed in a pop-up window and/or text message on a user device (e.g., “Probability of overnight hypoglycemia: 87%”). Alternatively or in combination, rather than displaying the actual probability value, the notification may instead display a qualitative risk level of overnight hypoglycemia (e.g., “High,” “Moderate,” or “Low”). The risk levels can be determined using any suitable probability threshold (e.g., “High” corresponding to a probability greater than or equal to 75%, “Moderate” corresponding to a probability within a range from 40% to 75%), “Low” corresponding to a probability less than 40%). The notification can also include a message with recommendations, educational information, encouragement, etc. (e.g., “A small snack before bed can reduce the chances of an overnight low”, etc.). In some embodiments, the probability may be recalculated if conditions change, such as if the user records any activity, food, medication, etc., later in the evening before going to sleep.

Some or all of the steps of the method 500 can performed on demand by the patient, e.g., if the patient requests a forecast of the overnight hypoglycemia risk before going to sleep. Alternatively, some or all of the steps of the method 500 can be performed automatically, such as if the user device is set to automatically request a forecast at a fixed time each evening (e.g., at a regular time set by the patient, at a time calculated from the patient's sleep patterns, a common bedtime for that or any patient for that or any day of a week). If calculated automatically (as opposed to on-demand), the forecast results may be sent to the patient every time, and/or when the probability of overnight hypoglycemia exceeds a threshold set by the system and/or by the patient.

### Graphical User Interfaces

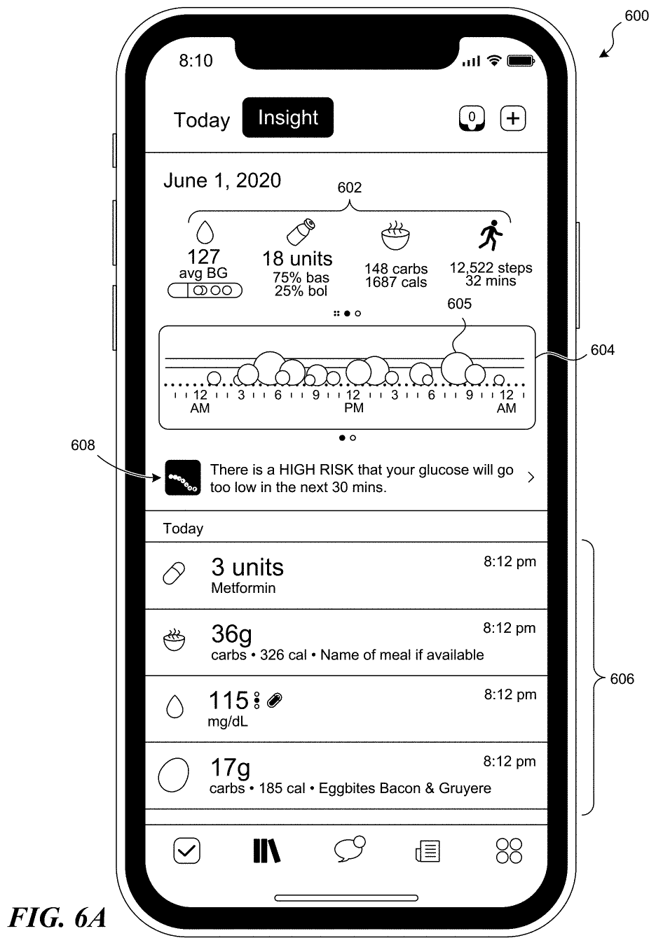

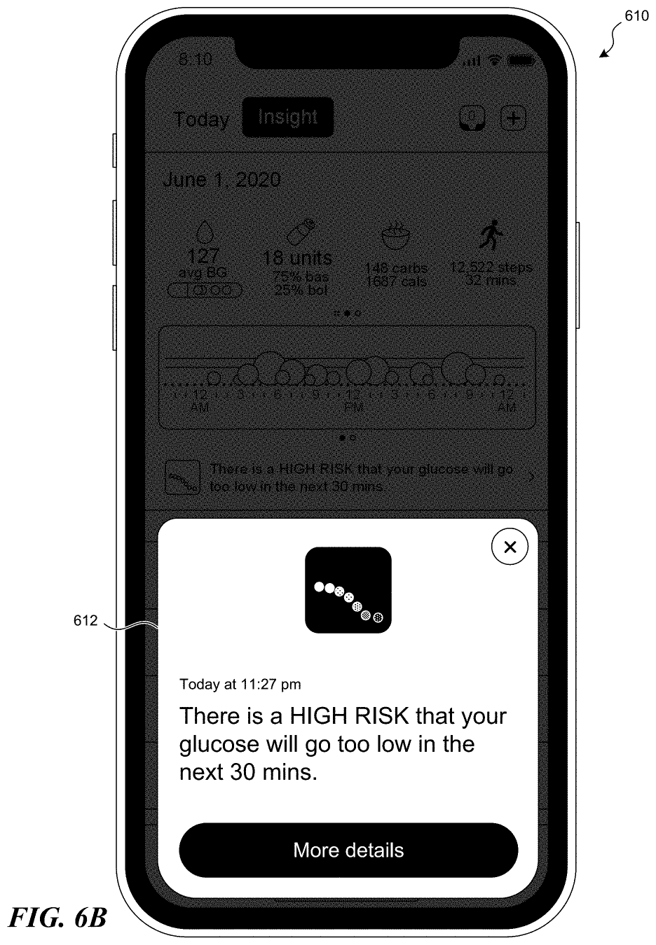

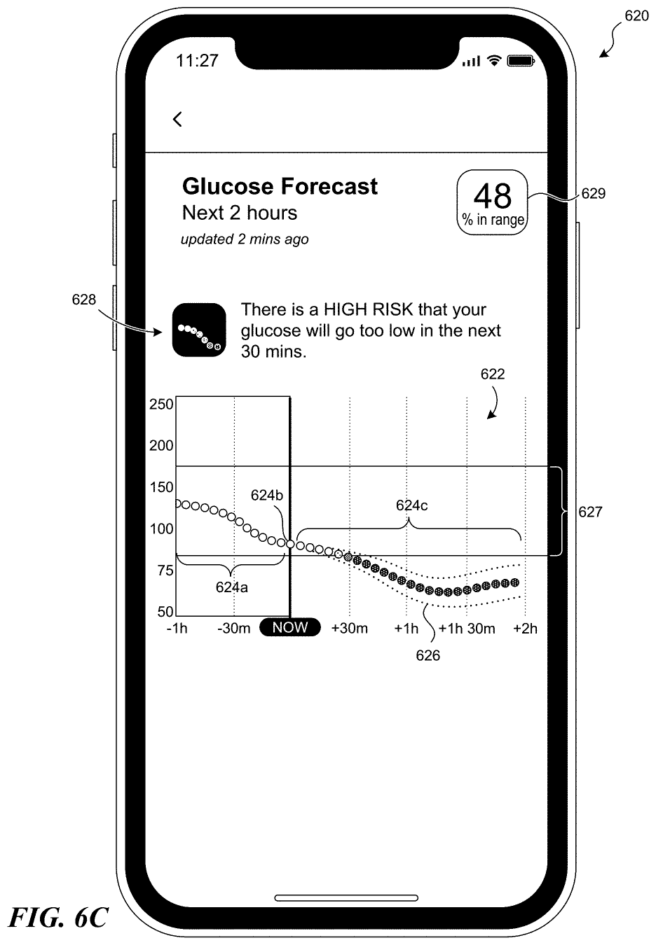

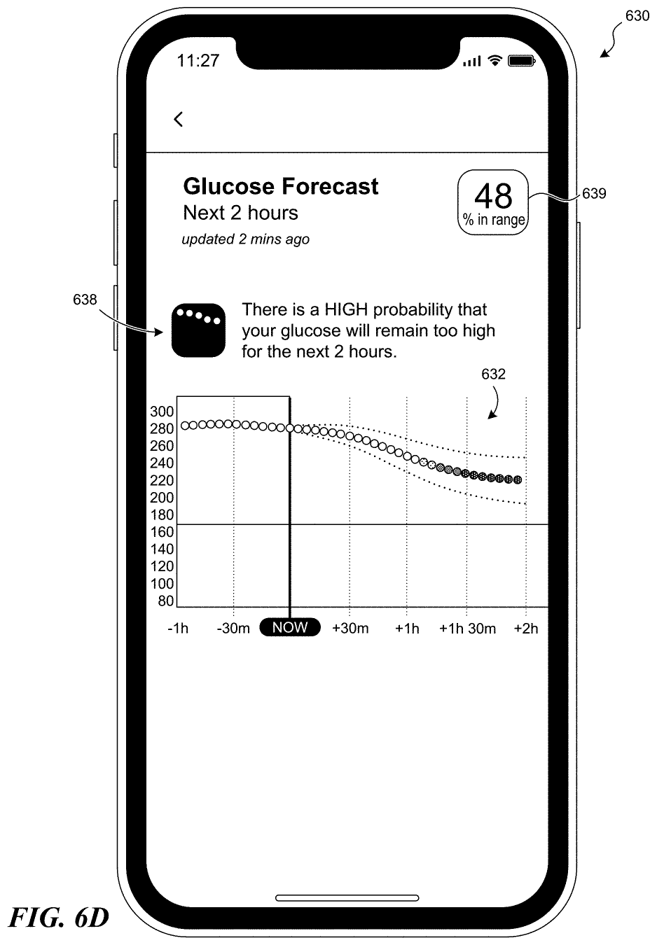

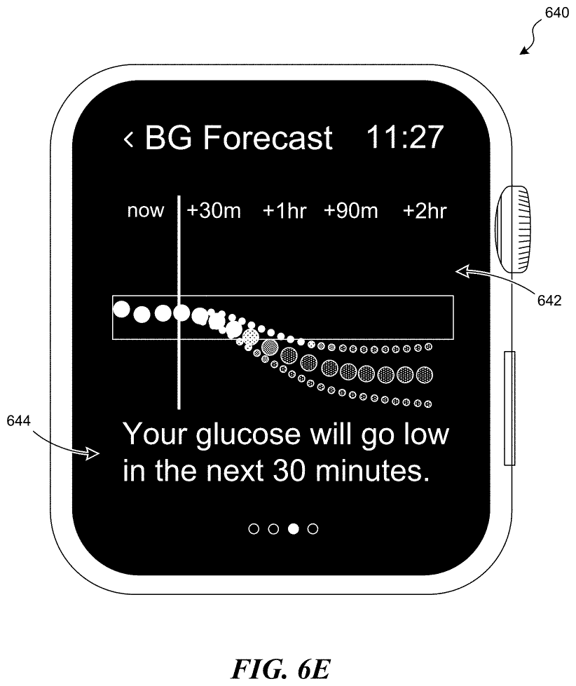

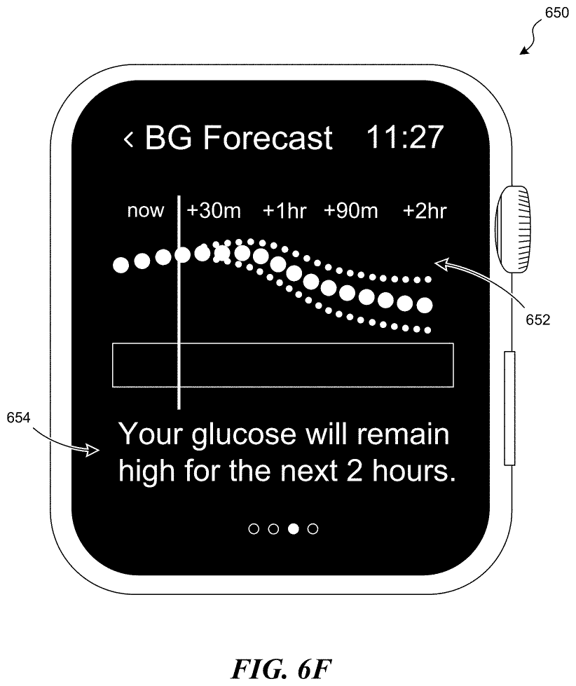

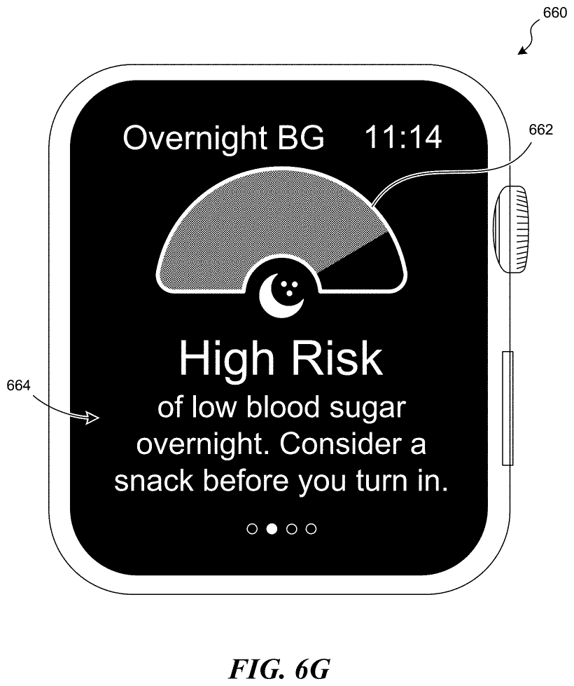

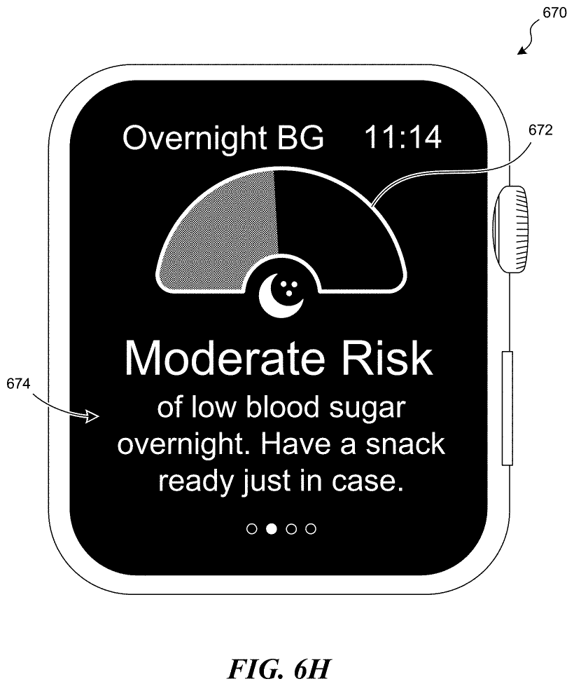

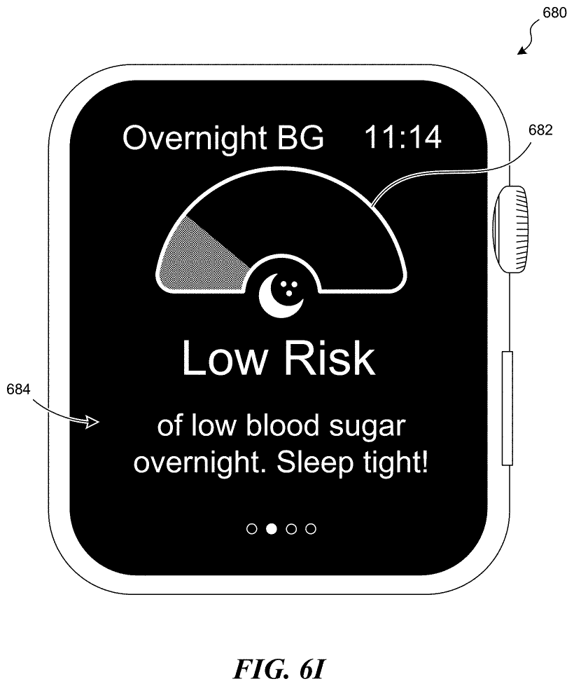

FIGS. 6A-6I illustrate various graphical user interfaces 600-680 configured in accordance with embodiments of the present technology. The graphical user interfaces 600-680 can be used with any of the processes or methods described herein with respect to FIGS. 1-5. The graphical user interfaces 600-680 can be generated by a software application (e.g., a mobile application or “app”) and displayed on a user device (e.g., user devices 104 of FIG. 1). For example, the graphical user interfaces 600-640 of FIGS. 6A-6D can be displayed on a mobile device (e.g., a smartphone, tablet computer), while the graphical user interfaces 640-680 of FIGS. 6E-6I can be displayed on a wearable device (e.g., a smartwatch). In other embodiments, however, the graphical user interfaces 600-680 can be adapted for display on other types of devices, such as a laptop computer, a personal computer, or any other suitable computing device. The graphical user interfaces 600-680 can be used to display information, notifications, recommendations, and/or other output produced by an analysis and/or forecasting system (e.g., system 102 of FIG. 1) to a patient or other user (e.g., a healthcare professional).

Referring first to FIG. 6A, the graphical user interface 600 (“interface 600”) can be configured for display on a smartphone or other mobile device. The graphical user interface 600 can provide an overview of the patient's health status and activities. For example, the interface 600 can include a summary section 602 with icons, text, and/or other graphical elements indicating key patient data for the day. For example, the summary section 602 can display the average blood glucose level, the total amount of insulin taken (e.g., divided into basal and bolus amounts), the total amount of food consumed (e.g., in terms of total carbohydrates and/or total calories), and the total amount of physical activity (e.g., number of steps, exercise duration). The interface 600 can also include a timeline 604 with graphical elements 605 corresponding to various measurements and/or events that occurred within a 24-hour period (e.g., blood glucose measurements, insulin intake, meals, physical activity). The interface 600 can also include a feed 606 displaying information for the most recent measurements and/or events. The interface 600 can also display an alert notification 608 with information relevant to the patient's blood glucose state (e.g., whether the patient's blood glucose level is predicted to go too high or too low within a future time period). The content of the alert notification 608 can be based on predictions generated in accordance with the techniques described herein. The summary section 602, timeline 604, feed 606, and/or alert notification 608 can be continuously updated as new blood glucose measurements, new event data, and/or new predictions are received.

FIG. 6B illustrates a graphical user interface 610 (“interface 610”) for displaying an alert notification 612. The alert notification 612 can include a pop-up window, text, and/or other graphical elements indicating that the patient's predicted blood glucose state will be outside a predetermined range (e.g., above or below a threshold value) within a future time period (e.g., the next 15 minutes, 30 minutes, 60 minutes, 90 minutes, 2 hours, 4 hours). For example, in the illustrated embodiment, the alert notification 612 indicates that there is a high risk that the patient's blood glucose level will go too low within the next 30 minutes.

FIG. 6C illustrates a graphical user interface 620 (“interface 620”) for displaying a patient's blood glucose levels over time. The interface 620 can include a graph, timeline, chart, table, and/or other graphical elements displaying multiple blood glucose values. In the illustrated embodiment, for example, blood glucose values 624a-c are displayed on a graph 622 with time on the horizontal axis and blood glucose level on the vertical axis. The displayed blood glucose values can include past blood glucose values 624a (e.g., actual blood glucose measurements obtained over the last 15 minutes, 30 minutes, 60 minutes, 90 minutes, 2 hours, or 4 hours), the patient's current blood glucose value 624b (e.g., the most recent blood glucose measurement) and/or future blood glucose values 624c (e.g., predicted future blood glucose values for the next 15 minutes, 30 minutes, 60 minutes, 90 minutes, 2 hours, or 4 hours). Optionally, the future blood glucose values 624c may be displayed along with an envelope 626 or other graphical elements indicating the degree of uncertainty in the prediction. Each blood glucose value can be color-coded, shaded, or otherwise visually differentiated to indicate whether the value is within the normal range, too high, or too low. The graph can also include a band 627 or other graphical element indicating a normal or target range for blood glucose levels. The normal or target range may be a standardized range of blood glucose values, or may be customized for the particular patient (e.g., based on recommendations from a healthcare professional, the patient's particular conditions, etc.).

The interface 620 can also include an alert notification 628 if the patient's blood glucose levels are predicted to go outside of the normal range during a future period. In the illustrated embodiment, for example, the patient's blood glucose level is currently within the normal range, but is predicted to fall too low within the 30 minutes. Optionally, the interface 620 can also include a time in range notification 629 displaying the percentage of time over the forecast time period that the blood glucose levels are predicted to be within the normal or target range (e.g., from 70 mg/dL to 140 mg/dL).

FIG. 6D illustrates a graphical user interface 630 (“interface 630”) for displaying a patient's blood glucose levels over time. The interface 630 can be generally similar to the interface 620 of FIG. 6C. For example, the interface 630 can include a graph 632 of blood glucose levels over time and, optionally, an alert notification 638 and/or a time in range notification 639. In the illustrated embodiment, the alert notification 638 indicates that the patient's blood glucose level is currently too high and is predicted to remain too high over the next 2 hours.

FIG. 6E illustrates a graphical user interface 640 (“interface 640”) for displaying a patient's blood glucose levels over time. The interface 640 can be designed for display on a relatively small device, such as a smartwatch. Accordingly, the information presented on the interface 640 can be condensed and/or simplified compared to interfaces designed for display on larger devices. For example, the interface 640 can include a graph 642 of blood glucose levels over time that conveys similar information as the graph 622, but in a more compact format. For example, rather than displaying a vertical axis labeled with blood glucose levels, the blood glucose values shown in the graph 642 can be color-coded or otherwise visually distinguished to indicate whether they are high, low, or within normal range. The interface 640 can also include an alert notification 644 with information regarding the patient's predicted blood glucose state, if appropriate (e.g., the patient's blood glucose level is predicted to go too low within the next 30 minutes).

FIG. 6F illustrates a graphical user interface 650 (“interface 650”) for displaying a patient's blood glucose levels over time. The interface 650 can be generally similar to the interface 640 of FIG. 6E, except that the graph 652 and alert notification 654 indicate that the patient's blood glucose level is currently too high and is predicted to remain too high over the next 2 hours.

FIG. 6G illustrates a graphical user interface 660 (“interface 660”) for displaying an overnight hypoglycemia prediction. The interface 660 can include a graphical element 662 that visually depicts the probability that the patient will experience a hypoglycemia event overnight. The interface 660 can also include an alert notification 664 with information regarding the risk of an overnight hypoglycemia event (e.g., “high risk”), and optionally, a recommendation for an action or actions to mitigate the risk (e.g., consuming food before going to sleep).

FIG. 6H illustrates a graphical user interface 670 (“interface 670”) for displaying an overnight hypoglycemia prediction. Similar to the interface 660 of FIG. 6G, the interface 670 includes a graphical element 672 that visually depicts the probability of experiencing hypoglycemia while sleeping, as well as an alert notification 674 with information regarding the risk of overnight hypoglycemia (e.g., “moderate risk”). In the illustrated embodiment, the alert notification 674 also includes a recommendation for how the patient can reduce the risk of overnight hypoglycemia (e.g., having food nearby in case a hypoglycemia event occurs).

FIG. 6I illustrates a graphical user interface 680 (“interface 690”) for displaying an overnight hypoglycemia prediction. Similar to the interfaces 660, 670 of FIGS. 6G and 6H, the interface 680 includes a graphical element 682 that visually depicts the probability of experiencing hypoglycemia while sleeping, as well as an alert notification 684 with information regarding the risk of overnight hypoglycemia (e.g., “low risk”). In the illustrated embodiment, the alert notification 684 indicates that no further actions are needed to mitigate risk before going to sleep.

### Additional Embodiments

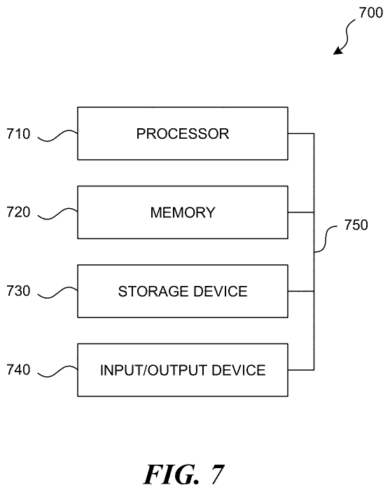

FIG. 7 is a schematic block diagram of a computing system or device (“system 700”) configured in accordance with embodiments of the present technology. The system 700 can be incorporated into or used with any of the systems and devices described herein, such as the system 102 and/or user devices 104 of FIG. 1. The system 700 can be used to perform any of the processes or methods described herein with respect to FIGS. 1-6I. The system 700 can include a processor 710, a memory 720, a storage device 730, and an input/output device 740. Each of the components 710, 720, 730 and 740 can be interconnected using a system bus 750. The processor 710 can be configured to process instructions for execution within the system 700. In some implementations, the processor 710 can be a single-threaded processor. In alternate implementations, the processor 710 can be a multi-threaded processor. The processor 710 can be further configured to process instructions stored in the memory 720 or on the storage device 730, including receiving or sending information through the input/output device 740. The memory 720 can store information within the system 700. In some implementations, the memory 720 can be a computer-readable medium. In alternate implementations, the memory 720 can be a volatile memory unit. In yet some implementations, the memory 720 can be a non-volatile memory unit. The storage device 730 can be capable of providing mass storage for the system 700. In some implementations, the storage device 730 can be a computer-readable medium. In alternate implementations, the storage device 730 can be a floppy disk device, a hard disk device, an optical disk device, a tape device, non-volatile solid state memory, or any other type of storage device. The input/output device 740 can be configured to provide input/output operations for the system 700. In some implementations, the input/output device 740 can include a keyboard and/or pointing device. In alternate implementations, the input/output device 740 can include a display unit for displaying graphical user interfaces.

In some embodiments, the current subject matter relates to a computer-implemented method for forecasting and interpreting blood glucose concentration for a user using various data, including continuous glucose monitoring data of the user and/or any other users. The method may include receiving input data (e.g., user data, aggregated data that may include blood glucose concentrations, physical activity, etc., time related data, time-stamped features, etc.), transforming and aggregating the received input data, generating predictions, combining predictions with pooled user data (e.g., from the user and/or other users) to generate a model, training the model, and applying the model to generate predictions based on the model. In some embodiments, the prediction data may be interpreted and feedback may be provided to the user (e.g., displayed on a user interface).

In some embodiments, the current subject matter can provide a method for determining forecasts of a user's blood glucose concentration at a point in the future from 15-minutes to 24-hours (in exemplary, non-limiting embodiments, the intervals may be 30 minutes to 8 hours; up to 12 hours, and/or any desired period of time), quantifying confidence bounds on the forecasted data, and producing an interpretation of whether the forecast is above, below or within the range consistent with any given target blood glucose health (al c) goal. For forecasting purposes, the current subject matter can use past blood glucose concentration values, grams of carbohydrates eaten at meals, workouts or minutes of activity, past values of weight, past values of al c, year of diagnosis, etc., and/or any combination thereof. It can also use the above information that users have entered, which can widely vary from user to user, and from month to month for a given user.

In some embodiments, the current subject matter relates to a computer-implemented method for predicting occurrence of a hypoglycemic event during a predetermined period of time for a user using various data, including data of the user and/or any other users. The method may include receiving input data (e.g., user's data, aggregated data that may include sleep data, heart rate data, blood glucose concentrations, physical activity, etc., time-related data, time-stamped features, etc.), transforming and aggregating the received input data, generating predictions, combining predictions with pooled user data (e.g., from the user and/or other users) to generate a model, training and testing the model, and applying the model to generate predictions of an occurrence of a hypoglycemic event during a predetermined period of time based on the model. In some embodiments, the predictions data may be interpreted and a feedback may be provided to the user (e.g., displayed on a user interface).

Non-transitory computer program products (i.e., physically embodied computer program products) are also described that store instructions that, when executed by one or more data processors of one or more computing systems, cause at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g., the Internet, a wireless wide area network, a local area network, a wide area network, a wired network, or the like), via a direct connection between one or more of the multiple computing systems, etc.

The systems and methods disclosed herein can be embodied in various forms including, for example, a data processor, such as a computer that also includes a database, digital electronic circuitry, firmware, software, or in combinations of them. Moreover, the above-noted features and other aspects and principles of the present disclosed implementations can be implemented in various environments. Such environments and related applications can be specially constructed for performing the various processes and operations according to the disclosed implementations or they can include a general-purpose computer or computing platform selectively activated or reconfigured by code to provide the necessary functionality. The processes disclosed herein are not inherently related to any particular computer, network, architecture, environment, or other apparatus, and can be implemented by a suitable combination of hardware, software, and/or firmware. For example, various general-purpose machines can be used with programs written in accordance with teachings of the disclosed implementations, or it can be more convenient to construct a specialized apparatus or system to perform the required methods and techniques.

The systems and methods disclosed herein can be implemented as a computer program product, i.e., a computer program tangibly embodied in an information carrier, e.g., in a machine readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

These computer programs, which can also be referred to programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user can provide input to the computer. Other kinds of devices can be used to provide for interaction with a user as well. For example, feedback provided to the user can be any form of sensory feedback, such as for example visual feedback, auditory feedback, or tactile feedback; and input from the user can be received in any form, including, but not limited to, acoustic, speech, or tactile input.

The technology described herein can be implemented in a computing system that includes a back-end component, such as for example one or more data servers, or that includes a middleware component, such as for example one or more application servers, or that includes a front-end component, such as for example one or more client computers having a graphical user interface or a Web browser through which a user can interact with an implementation of the subject matter described herein, or any combination of such back-end, middleware, or front-end components. The components of the system can be interconnected by any form or medium of digital data communication, such as for example a communication network. Examples of communication networks include, but are not limited to, a local area network (“LAN”), a wide area network (“WAN”), and the Internet.

The computing system can include clients and servers. A client and server are generally, but not exclusively, remote from each other and typically interact through a communication network. The relationship of client and server arises by virtue of computer programs running on the respective computers and having a client-server relationship to each other.

### EXAMPLES

The following examples are included to further describe some aspects of the present technology, and should not be used to limit the scope of the invention.

### Example 1: Hypo- and Hyperglycemia Prediction from Pooled CGM Data

The present example provides a method for using self-care data and blood glucose data from CGM devices collected from thousands of individuals via a mobile app to make and assess retrospective predictions of low or high blood glucose. Patients used the mobile app to enter food, medication physical activity, and other self-care data, as well as personal data such as gender and year of diagnosis. The mobile app was also used to passively read CGM-collected blood glucose values.

The data for this example included over 10 million hours of CGM data as well as self-care data from a sample of over 3000 users. Data used for this study included over 10 million hours of CGM data as well as self-care data from a sample of over 3000 users. The patients whose data was used in the study were 88% type 1 (T1D/LADA), 9% type 2, and 3% unreported; 33% were males, 26% females, and 41% unreported.

Data was partitioned into a training set, including data from a random subsample of users, and a test set (all remaining data). The training values were used to train a supervised learning model. The model was applied to the test set data to generate retrospective predictions of each user's blood glucose 30 and 60 minutes into the future. The predicted values were then compared to the recorded test set CGM values.

In addition, related models were trained on the same training data, to predict the probabilities that blood glucose levels would be low (<70 mg/dL) or high (>180 mg/dL) in the next 30 minutes, next hour, and next 4 hours. The probability of a low or high blood glucose exceeding a threshold value was interpreted as an “alert” for likely hypo- or hyperglycemia. The accuracy of the alerts against the presence or absence of high or low events in subsequent CGM values, evaluating the precision (percent of alerts that correctly identified upcoming events), recall (percent of actual events that were preceded by alerts) and area under the receiver operating characteristic curve (AUC), which represents the severity of the trade-off between precision and recall as the alert threshold is varied. AUCs approaching the maximum value of 100% indicate greater accuracy.

Each prediction was based not only on past observations from the user being predicted, but also on all observations in the training set data collected prior to the point in historical time from which the forecast was calculated.

Table 3 below shows the blood glucose prediction accuracy at 30-minute and 1-hour horizons. The mean absolute relative deviation (MARD) for 30-minute predictions was 4.3%, with 98.7% of predictions falling in Zone A of the Clarke Error Grid, and 99.9% in Zone A or B. The MARD for 60-minute predictions was 13.4%, with 79.4% in Zone A, and 98.4% in Zone A or B.

TABLE 3
Blood Glucose Prediction Accuracy
at 30-Minute and 1-Hour Horizons.

| Metric | 30-Minute Forecast | 1-Hour Forecast |
|---|---|---|
| Clarke A^{a} | 98.7% | 79.4% |
| Clarke A or B^{b} | 99.9% | 98.4% |
| <50 mg/dL^{c} | 99.9% | 92.2% |
| <30 | 98.9% | 78.7% |
| <20 | 95.8% | 63.9% |
| <10 | 80.4% | 38.4% |
| <5 | 54.6% | 20.3% |
| MARD^{d} | 4.3% | 13.4% |

^{a}Percent of predicted values landing in the “A” zone of the Clarke Error Grid. These predictions are within 20% of the measured value, if the measured value is over 70 mg/dL, or less than 70 mg/dL if the measured value is less than 70 mg/dL.
^{b}Percent of predicted values landing in either the “A” or “B” zone of the Clarke Error Grid.
^{c}Percent of prediction errors with absolute value of less than 50 mg/dL.
^{d}Mean Absolute Relative Deviation: 100 × |prediction − actual|/actual. For comparison, CGM measurements vs. lab-measured blood glucose values typically have MARDs of 9-10%.

Table 4 below shows accuracies of the hyperglycemia and hypoglycemia predictions. Hypoglycemia predictions showed 93.2% recall, 89.4% precision, and 99.5% AUC at 30 minutes; 83.2% recall, 74.1% precision and 98.6% AUC at 1 hour, and 62% precision, 84% recall and 91.9% AUC at 4 hours. Hyperglycemia predictions showed 98.9% recall, 97.6% precision and 99.5% AUC at 30 minutes; 95.0% recall, 92.6% precision, and 98.6% AUC at 1 hour; and 83.6% re call, 83.8% precision and 91.6% AUC at 4 hours.

TABLE 4
Accuracy of Hypo- (“Low”) and Hyperglycemia (“High”) Alerts

| Metric | Within 30 minutes | Within 1 hour | Within 4 hours |
|---|---|---|---|
| Low - Recall | 93.2% | 83.2% | 62.0% |
| Low - Precision | 89.4% | 74.1% | 84.0% |
| Low - AUC | 99.8% | 98.6% | 91.9% |
| High - Recall | 98.9% | 95.0% | 83.6% |
| High - Precision | 97.6% | 92.6% | 83.8% |
| High - AUC | 99.9% | 98.8% | 91.6% |

These results demonstrate that pooling blood glucose data from thousands of users can allow for accurate predictions of hypo- and hyperglycemia up to 4 hours in advance. Such predictions can potentially inform proactive, preventative self-care.

### Example 2: Overnight Hypoglycemia Prediction for CGM Users

The present example provides a method for using CGM data and self-care data collected via a mobile app to retrospectively predict the occurrence of overnight hypoglycemia. The data used included over 560,000 person-nights of blood glucose data, self-care data (e.g., medication, food, physical activity, sleep), and personal data (e.g., gender, year of diagnosis) from over 3000 app users with CGM devices. Data were identified as “overnight” by comparison to sleep data (when available), and otherwise by assuming an overnight period from 11 PM to 6 AM in each user's local time zone. Users in the sample were diagnosed with type 1/LADA (86%), type 2 (8%), and unreported (6%); 28% of users were diagnosed in the past 5 years.

Data were divided into a training set comprising about 360,000 nights before a selected date, and a test set comprising about 200,000 nights after the selected date. Training data were used to train two machine learning models. Both were pooled models, meaning that each prediction was based on the data from all users, not just on data from the person for which the prediction was being made.

The first model was trained to predict, as of bedtime, the probability of a hypoglycemic event (defined for the purposes of this example as any blood glucose level less than 70 mg/dL) occurring subsequently during the night. The trained model was then used to predict the test set nights, comparing each predicted probability to whether or not there was actually a hypoglycemic event. Hypoglycemia was considered “likely” if the predicted probability was above a set threshold probability. Higher thresholds may give fewer false alarms, but may also result in more missed events. The model was evaluated by the area under the receiver operating characteristic curve (AUC), which characterizes the degree to which false alarms can be reduced without missing events. A greater AUC indicates greater accuracy. The training set results were also analyzed to determine if criteria existed that could identify, at bedtime, nights that could be predicted more accurately.

As an alternative approach, a second model was trained to predict—again, as of bedtime—the minimum blood glucose value for the coming night. A predicted minimum of less than 70 mg/dL was interpreted as a prediction of hypoglycemia.

Table 5 illustrates a comparison of predicted probabilities to actual frequencies of overnight hypoglycemia for the test set nights. Test set nights were grouped by predicted probability of hypoglycemia, and the predictions were compared to the actual frequency of hypoglycemia in each group, As can be seen in Table 5 below, the predicted probabilities were consistent with the actual frequencies of overnight hypoglycemia

TABLE 5
Predicted Probabilities and Actual Frequencies
of Overnight Hypoglycemia

| Predicted Probability | Actual Frequency |
|---|---|
| 0 to 10% | 4.6% |
| 10 to 20% | 14.9% |
| 20 to 30% | 24.6% |
| 30 to 40% | 36.3% |
| 40 to 50% | 45.2% |
| 50 to 60% | 53.5% |
| 60 to 70% | 63.5% |
| 70 to 80% | 72.9% |
| 80 to 90% | 84.2% |
| 90 to 100% | 97.5% |

Table 6 illustrates the accuracy of prediction results from the full test set. As shown in Table 6 below, the AUC for all predictions 82.2%. By examining the training set results, it was discovered that certain combinations of blood glucose variability, physical activity, food, and heart rate observed at bedtime indicated the prediction would be of higher accuracy. Approximately 30% of predictions in the test set met those criteria (“high confidence predictions”); the AUC for those predictions was 87.0%.

Predictions of minimum overnight blood glucose value had a test set mean absolute relative deviation (MARD) of 18.6%. High confidence predictions (the same 30% of test set nights described above) had a MARD of 15.4%.

TABLE 6
Test Set Prediction Results

| | All Predictions | High Confidence Predictions |
|---|---|---|
| Probability of | 82.2% | 87.0% |
| Hypoglycemia (AUC) | | |
| Minimum Overnight | 18.6% | 15.4% |
| Blood Glucose (MARD) | | |

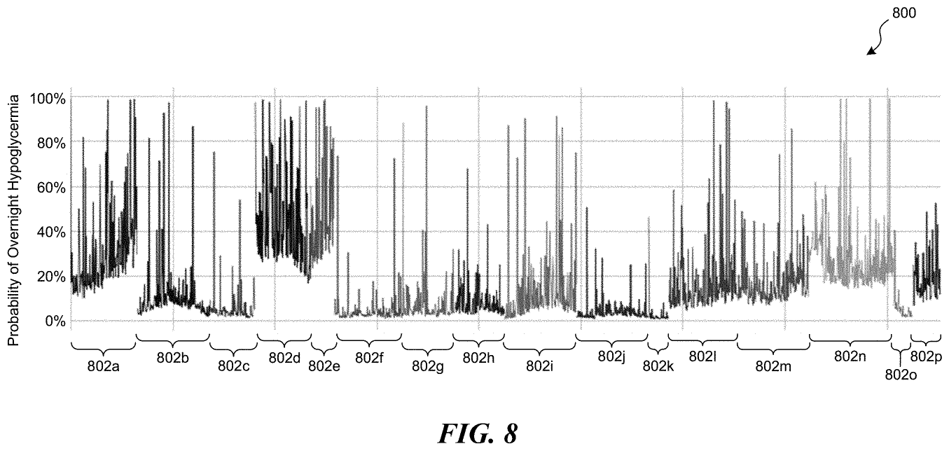

FIG. 8 is a graph 800 illustrating exemplary sequences of nightly probabilities of overnight hypoglycemia for 16 randomly selected individuals 802a-p. The graph 800 shows, for each individual, probability values calculated for a sequence of multiple nights, with one probability value per night. As can be seen in FIG. 8, variability is evident, not only from person to person, but also from night to night for a given person. While some individuals are consistently unlikely to experience overnight hypoglycemia, model-predicted probabilities are likely to be informative for those whose probability changes significantly from night to night.

These results demonstrate that pooling sleep, blood glucose, behavioral, and self-care data from thousands of users can accurately predict the probability of overnight hypoglycemia. Having such predictions at bedtime can facilitate preventative action, reduce concern, and improve both sleep and quality of life.

### CONCLUSION

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations can be within the scope of the following claims.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

As used herein, the phrase “and/or” as in “A and/or B” refers to A alone, B alone, and A and B.

As used herein, the term “user” can refer to any entity including a person or a computer.

Although ordinal numbers such as first, second, and the like can, in some situations, relate to an order; as used in this document ordinal numbers do not necessarily imply an order. For example, ordinal numbers can be merely used to distinguish one item from another. For example, to distinguish a first event from a second event, but need not imply any chronological ordering or a fixed reference system (such that a first event in one paragraph of the description can be different from a first event in another paragraph of the description).

From the foregoing, it will be appreciated that specific embodiments of the invention have been described herein for purposes of illustration, but that various modifications may be made without deviating from the scope of the invention. Accordingly, the invention is not limited except as by the appended claims.

What is claimed is:

1. A computer-implemented method for forecasting a blood glucose state of a patient, the method comprising:
receiving blood glucose data of the patient;
generating at least one initial prediction of the blood glucose state by inputting the blood glucose data into a first set of machine learning models;
determining a plurality of features at least partly from the at least one initial prediction; and
generating a final prediction of the blood glucose state by inputting the plurality of features into a second set of machine learning models.

2. The computer-implemented method of claim 1, wherein:
the first set of machine learning models comprises a patient-specific model and a population model, wherein the patient-specific model is trained on previous blood glucose data of the patient, and wherein the population model is trained on a plurality of blood glucose data sets from a plurality of patients; and
the second set of machine learning models comprises an aggregate model trained on features from the plurality of blood glucose data sets from the plurality of patients.

3. The computer-implemented method of claim 2, wherein the plurality of features are determined from a first prediction generated by the patient-specific model, a second prediction generated by the population model, and the blood glucose data.

4. The computer-implemented method of claim 1, wherein the blood glucose data is generated by a continuous blood glucose monitoring device.

5. The computer-implemented method of claim 1, wherein the blood glucose data is correlated with at least one event, the at least one event including one or more of an insulin intake event, a food intake event, or a physical activity event.

6. The computer-implemented method of claim 1, wherein the first set of machine learning models comprises two or more different machine learning models, and wherein the at least one initial prediction comprises two or more initial predictions.

7. The computer-implemented method of claim 1, wherein the first set of machine learning models comprises a patient-specific model that is trained on previous blood glucose data of the patient.

8. The computer-implemented method of claim 1, wherein the first set of machine learning models comprises a population model that is trained on a plurality of blood glucose data sets from a plurality of patients.

9. The computer-implemented method of claim 1, wherein the second set of machine learning models comprises an aggregate model that is trained on features from a plurality of blood glucose data sets from a plurality of patients.

10. The computer-implemented method of claim 9, wherein the aggregate model is trained on features from one or more of personal data, event data, or prediction data for the plurality of patients.

11. The computer-implemented method of claim 1, wherein the plurality of features are determined at least partly from one or more of the blood glucose data, previous blood glucose data of the patient, or personal data of the patient.

12. The computer-implemented method of claim 1, wherein the initial prediction of the blood glucose state comprises a prediction of a blood glucose level.

13. The computer-implemented method of claim 1, wherein the final prediction of the blood glucose state comprises a prediction of one or more of a blood glucose level, a hypoglycemia event, or a hyperglycemia event.

14. A system for predicting a blood glucose state of a patient, the system comprising:
one or more processors; and
a memory storing instructions that, when executed by the one or more processors, cause the system to perform operations comprising:
receiving a plurality of blood glucose measurements of the patient, wherein at least some of the plurality of blood glucose measurements are associated with event data;
generating a first set of predictions of the blood glucose state using the plurality of blood glucose measurements and a first set of machine learning models;
generating feature data at least partly from the first set of predictions; and
generating a second set of predictions of the blood glucose state using the feature data and a second set of machine learning models.

15. The system of claim 14, further comprising a blood glucose sensor operably coupled to the one or more processors, wherein the blood glucose sensor is configured to generate the plurality of blood glucose measurements.

16. The system of claim 15, wherein the blood glucose sensor is a continuous blood glucose monitoring device.

17. The system of claim 15 further comprising a user device operably coupled to the one or more processors, wherein the event data is received from the user device.

18. The system of claim 17, wherein the user device is a wearable device, a mobile device, or a sensor.

19. The system of claim 15, wherein the event data comprises one or more of insulin data, meal data, or physical activity data.

20. The system of claim 15, wherein the first set of machine learning models comprises an individualized machine learning model and a population machine learning model.

21. The system of claim 15, wherein the second set of machine learning models comprises an aggregate model that is trained on feature data generated from a plurality of patient data sets.

22. The system of claim 15, wherein the feature data is generated at least partly from one or more of the plurality of blood glucose measurements, previous blood glucose measurements of the patient, or personal data of the patient.

23. The system of claim 15, further comprising a display operably coupled to the one or more processors, wherein the display is configured to output a notification to a user.

24. The system of claim 23, wherein the notification comprises one or more of a predicted blood glucose level, a predicted likelihood of a hypoglycemia event, or a predicted likelihood of a hyperglycemia event.

25. A non-transitory computer-readable storage medium including instructions that, when executed by a computing system, cause the computing system to perform operations comprising:
receiving blood glucose data and event data of a patient;
generating a first prediction of a future blood glucose state of the patient by inputting the blood glucose data and event data into a patient-specific machine learning model, wherein the patient-specific machine learning model is trained on previous blood glucose data and previous event data of the patient;
generating a second prediction of the future blood glucose state by inputting the blood glucose data and the event data into a population machine learning model, wherein the population machine learning model is trained on blood glucose data and event data of a plurality of patients;
determining a plurality of features from the first prediction, the second prediction, the blood glucose data of the patient, and the event data of the patient; and
generating a final prediction of the future blood glucose state by inputting the plurality of features into an aggregate machine learning model, wherein the aggregate machine learning model is trained on features extracted from the blood glucose data and the event data of the plurality of patients.
